(19) United States
(12) Patent Application Publication
(10) Pub. No.: US 2026/0335348 A1
(43) Pub. Date: Oct. 29, 2026

(54) PHARMACEUTICAL FORMS OF A CD73 INHIBITOR

(71) Applicant: ORIC Pharmaceuticals, Inc., South San Francisco, CA (US)

(72) Inventors: Mohammad AL-SAYAH, South San Francisco, CA (US); Sandy KOPPENOL, San Mateo, CA (US); Archana KUMAR, San Jose, CA (US); Ajit Singh NARANG, Belmont, CA (US); Robert Ronald MILBURN, South San Francisco, CA (US)

(21) Appl. No.: 19/110,337

(22) PCT Filed: Sep. 13, 2023
(86) PCT No.: PCT/US2023/074031

§ 371 Date: Mar. 10, 2025

Related U.S. Application Data
(60) Provisional application No. 63/375,580, filed on Sep. 14, 2022.
(60) Provisional application No. 63/497,323, filed on Apr. 20, 2023.

(51) Int. Cl. A61K 31/7064; A61K 9/20; C07C 55/10; C07C 65/05; C07F 9/6561

(52) U.S. Cl. CPC A61K 31/7064; A61K 9/2009; A61K 9/2013; A61K 9/2027; A61K 9/2054; C07C 55/10; C07C 65/05; C07F 9/6561

(57) ABSTRACT

The present disclosure relates to pharmaceutical forms of a CD73 inhibitor, polymorphic forms of the pharmaceutical forms, pharmaceutical compositions comprising the pharmaceutical forms, and methods of treating subjects having cancer by administering one of the pharmaceutical forms or a pharmaceutical composition comprising one of the disclosed pharmaceutical forms.

### CROSS-REFERENCE

This application claims the benefit of U.S. Provisional Application Ser. No. 63/375,580 filed Sep. 14, 2022 and U.S. Provisional Application Ser. No. 63/497,323 filed Apr. 20, 2023; which are hereby incorporated by reference in their entirety.

### BACKGROUND OF THE INVENTION

CD73 is a glycosylphosphatidylinositol (GPI) anchored cell surface protein that catalyzes the hydrolysis of AMP to adenosine, and works in concert with CD39, which converts ATP into AMP. The resulting adenosine functions as a signaling molecule that activates the P1 receptors expressed on the cell surface in many different tissues. Four G protein-coupled P1 or adenosine receptors have been cloned and designated as A1, A2A, A2B, and A3. Adenosine impacts a wide range of physiological processes including neural function, vascular perfusion, and immune responses. In doing so, this metabolite regulates CNS, cardiovascular, and immune system functions, to name a few.

Increasing evidence suggests that interactions between tumor cells and their microenvironment are essential for tumorigenesis. The purinergic signaling pathway in which CD73 plays a critical role, has emerged as an important player in cancer progression. It has become clear in recent years that adenosine is one of the most important immunosuppressive regulatory molecules in the tumor microenvironment, and contributes to immune escape and tumor progression.

CD73 is a key protein molecule in cancer development. CD73 has been found to be overexpressed in many cancer cell lines and tumor types including, for example, breast cancer, colorectal cancer, ovarian cancer, gastric cancer, gallbladder cancer, and cancers associated with poor prognosis.

The expression of CD73 in tumors is regulated by a variety of mechanisms. CD73 expression is negatively regulated by estrogen receptor (ER) in breast cancer. Thus, CD73 is highly expressed in ER negative breast cancer patients. The hypoxia-inducible factor-1α (HIF-1α) has also been shown to regulate CD73 transcription. Additionally, inflammatory factors such as IFN-γ affect CD73 levels. CD73 expression is also epigenetically regulated by CpG island methylation in cell lines and clinical tumor samples.

In addition to being a prognostic biomarker in cancer patients, overexpression of CD73 has also been found to be functionally linked to therapy resistance. Elevated levels of CD73 were initially linked to resistance to a variety of chemotherapeutic agents including vincristine and doxorubicin.

CD73 has also been shown to be involved in immunotherapy resistance. This ectonucleotidase participates in the process of tumor immune escape by inhibiting the activation, clonal expansion, and homing of tumor-specific T cells (in particular, T helper and cytotoxic T cells); impairing tumor cell killing by cytolytic effector T lymphocytes; driving, via pericellular generation of adenosine, the suppressive capabilities of Treg and Th17 cells; enhancing the conversion of type 1 macrophages into tumor-promoting type 2 macrophages; and promoting the accumulation of MDSCs.

Small molecular inhibitors and monoclonal antibodies targeting CD73 have shown anti-tumor activity in a variety of immune-competent but not in immune-deficient mouse tumor models. Overall, these studies suggest that anti-CD73 therapy activity is dependent on its ability to elicit immune responses in vivo.

Antibodies which block PD-1, PD-L1, and CTLA-4 have shown impressive objective response in cancer patients. Recent data demonstrates that anti-CD73 mAb significantly enhances the activity of both anti-CTLA-4 and anti-PD-1 mAbs in several mouse tumor models. In addition to checkpoint blockade, CD73-mediated production of adenosine could contribute to resistance to additional immunotherapy modalities including CAR-T cells and cancer vaccines.

Interfering with CD73 activity represents a strategy to re-sensitize tumors to therapy. Based on the link between CD73 and therapy resistance, combining anti-CD73 treatment with chemotherapy or immunotherapy is an effective approach to enhance their activity in cancer patients with high CD73 levels. In some instances, CD73 expression serves as a biomarker to identify patients that could benefit from anti-CD73 combination therapy.

In some instances, the CD39/CD73 couple turns ATP-driven pro-inflammatory cell activity toward an adenosine-mediated anti-inflammatory state. A number of studies have shown changes in the activity of the CD39/CD73 axis during infections induced by a variety of microorganisms. An increase in CD73 expression has also been observed in the brain of mice infected with Toxoplasma gondii, which promotes the parasite life cycle through the production of adenosine. Thus, the pharmacological blockade of CD73 is a promising therapeutic approach to treat human toxoplasmosis.

Enhanced expression and activity of CD39 and CD73 have been observed in endothelial cells infected with cytomegalovirus (CMV). The increase in local adenosine production, associated with the upregulation of ecto-nucleotidases, generates an immunosuppressive and antithrombotic microenvironment, which facilitates viral entry into target cells.

In some instances, inhibitors of CD73, by driving a decrease on adenosine production, have applications as antiviral agents. The elevated expression/activity of CD39 and CD73 on lymphocytes of individuals infected with human immunodeficiency virus (HIV) indicates a role for ecto-nucleotidases in the immune dysfunction associated with this disease. In fact, an increased proportion of Tregs expressing CD39, as well as a positive correlation between CD39 expression on Tregs and disease progression has been observed in different cohorts of HIV-infected patients. It has also been shown that HIV-positive patients had a higher number of CD39+ Treg, and that their Teff exhibited an increased sensitivity in vitro to the suppressive effect of adenosine, which was related to the elevated expression of immunosuppressive A2A receptors.

In the central nervous system, adenosine plays a critical role in controlling a multitude of neural functions. Through the activation of P1 receptors, adenosine is involved in diverse physiological and pathological processes such as regulation of sleep, general arousal state and activity, local neuronal excitability, and coupling of the cerebral blood flow to the energy demand. In some instances, manipulation of adenosine production via CD73 inhibitors is useful for treating neurodegenerative diseases such as Alzheimer's disease, Parkinson's disease and Huntington's disease, and psychiatric disorders such as schizophrenia and autism.

Thus, there exists a need for the development of CD73 inhibitors, and pharmaceutical compositions comprising them, that can be advantageously used to treat cancers and infections in subjects in need thereof.

### SUMMARY OF THE INVENTION

Provided herein are pharmaceutically acceptable forms of Compound 1:

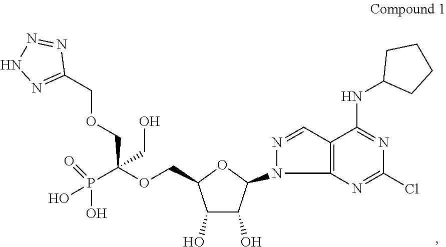 [SMILES: O=P(O)(O)[C@@](CO)(COCc1nn[nH]n1)OC[C@H]1O[C@@H](n2ncc3c(NC4CCCC4)nc(Cl)nc32)[C@H](O)[C@@H]1O]

wherein the pharmaceutically acceptable form is selected from a gentisate form and a succinate form. Also provided herein are polymorphic forms of a gentisate form and a succinate form of Compound 1. In some embodiments, a gentisate form comprises a gentisate salt. In some embodiments, a gentisate form comprises a gentisate co-crystal. In some embodiments, a gentisate form comprises a salt and a co-crystal. In some embodiments, a succinate form comprises a succinate salt. In some embodiments, a succinate form comprises a succinate co-crystal. In some embodiments, a succinate form comprises a salt and a co-crystal. Further provided herein are pharmaceutical compositions comprising amounts of a pharmaceutically acceptable form of Compound 1, wherein the pharmaceutically acceptable form is selected from a gentisate form and a succinate form. Some embodiments disclosed herein provide methods of inhibiting CD73 activity in a cell comprising contacting CD73 in the cell with an effective amount of a pharmaceutically acceptable form of Compound 1 as disclosed herein. Further provided herein are methods of treating cancer in a subject in need thereof, comprising administering to the subject a therapeutically effective amount of a pharmaceutically acceptable form of Compound 1 as disclosed herein. In some embodiments, the cancer is lung cancer, melanoma, breast cancer, ovarian cancer, colorectal cancer, gastric cancer, gallbladder cancer, prostate cancer, renal cancer, multiple myeloma, or a lymphoma. In other embodiments, the cancer expresses CD73. In still further embodiments are provided methods of treating cancer in a subject in need thereof, comprising administering to the subject a therapeutically effective amount of a pharmaceutically acceptable form of Compound 1 as disclosed herein, and one or more second therapeutic agents.

### DETAILED DESCRIPTION OF THE INVENTION

As used herein and in the appended claims, the singular forms “a,” “an,” and “the” include plural referents unless the context clearly dictates otherwise. Thus, for example, reference to “an agent” includes a plurality of such agents, and equivalents thereof known to those skilled in the art, and so forth. When ranges are used herein for physical properties, such as molecular weight, melting points, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Administering” when used in conjunction with a therapeutic means to administer a therapeutic systemically or locally, as directly into or onto a target tissue, or to administer a therapeutic to a subject whereby the therapeutic positively impacts the tissue to which it is targeted. Thus, as used herein, the term “administering,” when used in conjunction with a composition described herein, can include, but is not limited to, providing a composition into or onto the target tissue; providing a composition systemically to a subject by, e.g., oral administration whereby the therapeutic reaches the target tissue or cells. “Administering” a composition may be accomplished by injection, topical administration, and oral administration or by other methods alone or in combination with other known techniques.

The term “amorphous,” as used herein, refers to a solid composition having no measurable long-range order in the position of its molecules, as measured by analytical techniques known to those having ordinary skill in the art, such as x-ray powder diffraction (XRPD).

The terms “crystalline” and “crystallinity” refer to a solid composition having some measure of long-range order in the position of its molecules, as measured by analytical techniques known to those having ordinary skill in the art, such as x-ray powder diffraction (XRPD).

The term “differential scanning calorimetry,” as used herein means a method of thermal analysis described in USP <891>.

The terms “gentisate salt,” as used herein means a salt formed between Compound 1 and gentisic acid. The term “gentisic acid” as used herein means the compound having Chemical Abstracts Registry No. 490-79-9, the chemical name 2,5-dihydroxybenzoic acid, and the chemical structure below:

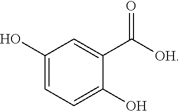 [SMILES: O=C(O)c1cc(O)ccc1O]

The term “gentisate,” as used herein refers to a form of Compound 1 in association with gentisic acid. As contemplated herein, a gentisate may be (a) a salt form comprising Compound 1 and gentisic acid, (b) a co-crystal comprising Compound 1 and gentisic acid, or (c) a mixture of a salt comprising Compound 1 and gentisic acid and a co-crystal comprising Compound 1 and gentisic acid.

The term “co-crystal” as used herein means a crystalline material comprising two or more different molecules, one of which is Compound 1, in a defined stoichiometric ratio within the same crystal lattice that are associated by nonionic and noncovalent bonds. In one embodiment is provided a co-crystal comprising Compound 1 and gentisic acid. In another embodiment is provided a co-crystal comprising compound 1 and succinic acid.

The term “pharmaceutically acceptable,” means the carrier, diluent or excipient must be compatible with the other ingredients of the formulation and not deleterious to the recipient thereof.

The term “pharmaceutical composition” shall mean a composition comprising one or more active ingredients, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “substantially amorphous,” as used herein, refers to a composition having little or no long range order in the position of its molecules. For example, substantially amorphous materials have less than about 15% crystallinity (e.g., less than about 10% crystallinity or less than about 5% crystallinity). As contemplated herein, the term substantially amorphous includes a composition which includes no measurable crystalline materials, as measured by analytical techniques known to those having ordinary skill in the art, such as x-ray powder diffraction (XRPD).

The term “succinate salt,” as used herein means a salt formed between Compound 1 and succinic acid. The term “succinic acid” as used herein means the compound having Chemical Abstracts Registry No. 110-15-6, the chemical names 1,2-ethanedicarboxylic acid and 1,4-butanedioic acid, and the chemical structure below:

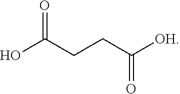

The term “succinate,” as used herein refers to a form of Compound 1 in association with succinic acid. As contemplated herein, a succinate may be (a) a salt form comprising Compound 1 and succinic acid, (b) a co-crystal comprising Compound 1 and succinic acid, or (c) a mixture of a salt comprising Compound 1 and succinic acid and a co-crystal comprising Compound 1 and succinic acid.

As used herein, the term “therapeutic” means an agent utilized to treat, combat, ameliorate, prevent, or improve an unwanted condition or disease of a subject.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

As used herein, the term “x-ray powder diffraction (XRPD)” means the technique of characterizing a solid for crystallinity or partial crystallinity by use of powder x-ray diffraction as set forth in USP <941>.

Provided herein is pharmaceutically acceptable form of Compound 1:

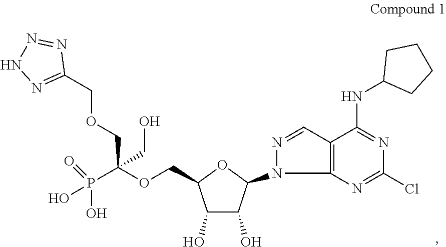 [SMILES: O=P(O)(O)[C@@](CO)(COCc1nn[nH]n1)OC[C@H]1O[C@@H](n2ncc3c(NC4CCCC4)nc(Cl)nc32)[C@H](O)[C@@H]1O]

wherein the pharmaceutically acceptable form is selected from a gentisate form and a succinate form.

In some embodiments is provided a pharmaceutically acceptable form of Compound 1, wherein the form is a gentisate form. In some embodiments, the gentisate form comprises a gentisate salt. In some embodiments, the gentisate form comprises a gentisate co-crystal. In some embodiments, the gentisate form comprises a salt and a co-crystal. In further embodiments is provided a gentisate form of Compound 1, wherein the molar ratio between Compound 1 and gentisic acid is about 1:1. In further embodiments is provided a gentisate form of Compound 1, wherein the molar ratio between Compound 1 and gentisic acid is about 2:1. In further embodiments is provided a gentisate form of Compound 1, wherein the molar ratio between Compound 1 and gentisic acid is about 1:2. In other embodiments is provided a gentisate form of Compound 1, wherein the gentisate form is a hydrate. In some embodiments, the hydrate of the gentisate form of Compound 1 is selected from a hemi-hydrate, a mono-hydrate, and a di-hydrate forms. In some embodiments, the hydrate is a hemi-hydrate. In some embodiments, the hydrate is a mono-hydrate. In some embodiments, the hydrate is a di-hydrate.

In further embodiments is provided a gentisate form of Compound 1, wherein the form is a solid. In some embodiments, such solid is crystalline. In some embodiments, such the crystalline solid exhibits a peak in an x-ray powder diffraction (XRPD) pattern at 9.25±0.2° 2-theta. In still further embodiments, the crystalline solid exhibits further peaks in an x-ray powder diffraction (XRPD) pattern at 6.97±0.2° 2-theta, 20.53±0.2° 2-theta, and 26.08±0.2° 2-theta. In still further embodiments, the crystalline solid exhibits further peaks in an x-ray powder diffraction (XRPD) pattern at 14.61±0.2° 2-theta and 18.89 0.2° 2-theta.

In a further embodiment is provided a gentisate form of Compound 1, wherein the form is a crystalline solid and exhibits a peak in an x-ray powder diffraction (XRPD) pattern at 9.3 0.2° 2-theta. In still further embodiments, the crystalline solid exhibits a further peak in an x-ray powder diffraction (XRPD) pattern at 7.1±0.2° 2-theta. In still further embodiments, the crystalline solid exhibits a further peak in an x-ray powder diffraction (XRPD) pattern at 20.1±0.2° 2-theta. In still further embodiments, the crystalline solid exhibits a further peak in an x-ray powder diffraction (XRPD) pattern at 19.0±0.2° 2-theta. In still further embodiments, the crystalline solid exhibits a further peak in an x-ray powder diffraction (XRPD) pattern at 26.2±0.2° 2-theta. In still further embodiments, the crystalline solid exhibits further peaks in an x-ray powder diffraction (XRPD) pattern at 4.8±0.2° 2-theta, 10.5±0.2° 2-theta, and 14.8±0.2° 2-theta.

In other embodiments is provided a crystalline solid of a gentisate form of Compound 1, wherein the crystalline form comprises a peak in a differential scanning calorimetry pattern of from about 150 °C to about 170 °C. In still further embodiments, the crystalline solids comprise a peak in a differential scanning calorimetry pattern of from about 150 °C to about 165 °C. In still further embodiments, the crystalline solids comprise a peak in a differential scanning calorimetry pattern of from about 161 °C to about 162 °C.

In other embodiments is provided a crystalline solid of a gentisate form of Compound 1, wherein the crystalline solid exhibit a loss in mass in a thermal gravimetric analysis of between about 1% to about 5% upon heating from about 31 °C to about 150 °C. In still further embodiments is provided a crystalline solid of a gentisate form of Compound 1, wherein the crystalline solid exhibit a loss in mass in a thermal gravimetric analysis of between about 3% to about 5% upon heating from about 31 °C to about 150 °C. In still further embodiments is provided a crystalline solid of a gentisate form of Compound 1, wherein the crystalline solid exhibits a loss in mass in a thermal gravimetric analysis of about 5% upon heating from about 31 °C to about 150 °C.

In other embodiments is provided a crystalline solid of a gentisate form of Compound 1, wherein the crystalline solid exhibits a solubility of at least 5 mg/mL in an aqueous solution having a pH of 1.7 and a temperature of 37 °C. In further embodiments is provided a crystalline solid of a gentisate form of Compound 1, wherein the crystalline solid exhibits a solubility of at least 10 mg/mL in an aqueous solution having a pH of 1.7 and a temperature of 37 °C. In further embodiments is provided a crystalline solid of a gentisate form of Compound 1, wherein the crystalline solid exhibits a solubility of at least 15 mg/mL in an aqueous solution having a pH of 1.7 and a temperature of 37 °C. In further embodiments is provided a crystalline solid of a gentisate form of Compound 1, wherein the crystalline solid exhibits a solubility of at least 20 mg/mL in an aqueous solution having a pH of 1.7 and a temperature of 37 °C. In further embodiments is provided a crystalline solid of a gentisate form of Compound 1, wherein the crystalline solid exhibits a solubility of at least 25 mg/mL in an aqueous solution having a pH of 1.7 and a temperature of 37 °C.

In other embodiments is provided a crystalline solid of the gentisate form of Compound 1, wherein the crystalline solid exhibits a solubility of between about 10 mg/mL and about 30 mg/mL in an aqueous solution having a pH of 1.7 and a temperature of 37 °C. In still further embodiments is provided a crystalline solid of a gentisate form of Compound 1, wherein the crystalline solid exhibits a solubility of between about 15 mg/mL and about 30 mg/mL in an aqueous solution having a pH of 1.7 and a temperature of 37 °C. In still further embodiments is provided a crystalline solid of a gentisate form of Compound 1, wherein the crystalline solid exhibits a solubility of between about 20 mg/mL and about 30 mg/mL in an aqueous solution having a pH of 1.7 and a temperature of 37 °C. In still further embodiments is provided a crystalline solid of a gentisate form of Compound 1, wherein the crystalline solid exhibits a solubility of between about 25 mg/mL and about 30 mg/mL in an aqueous solution having a pH of 1.7 and a temperature of 37 °C.

In other embodiments is provided a crystalline solid of a gentisate form of Compound 1, wherein the crystalline solids exhibit a solubility of at least 5 mg/mL in an aqueous solution having a pH of 2.5 and a temperature of 37 °C. In still further embodiments is provided a crystalline solid of a gentisate form of Compound 1, wherein the crystalline solid exhibits a solubility of at least 7.5 mg/mL in an aqueous solution having a pH of 2.5 and a temperature of 37 °C. In still further embodiments is provided a crystalline solid of a gentisate form of Compound 1, wherein the crystalline solid exhibits a solubility of at least 10 mg/mL in an aqueous solution having a pH of 2.5 and a temperature of 37 °C.

In other embodiments is provided a crystalline solid of a gentisate form of Compound 1, wherein the crystalline solid exhibits a solubility of at least 1 mg/mL in an aqueous solution having a pH of 4.4 and a temperature of 37 °C. In still further embodiments is provided a crystalline solid of a gentisate form of Compound 1, wherein the crystalline solid exhibits a solubility of at least 2.5 mg/mL in an aqueous solution having a pH of 4.4 and a temperature of 37 °C. In still further embodiments is provided a crystalline solid of a gentisate form of Compound 1, wherein the crystalline solid exhibits a solubility of at least 5 mg/mL in an aqueous solution having a pH of 4.4 and a temperature of 37 °C. In still further embodiments is provided a crystalline solid of a gentisate form of Compound 1, wherein the crystalline solid exhibits a solubility of at least 7.5 mg/mL in an aqueous solution having a pH of 4.4 and a temperature of 37 °C.

In other embodiments is provided a crystalline solid of a gentisate form of Compound 1, wherein the crystalline solid exhibits less than about 10% degradation of the total amount of a gentisate form of Compound 1 when the pharmaceutically acceptable form is stored at 5 °C for at least 7 days. In other embodiments is provided a crystalline solid of a gentisate form of Compound 1, wherein the crystalline solid exhibits less than about 9%, or less than about 8%, or less than about 7%, or less than about 6%, or less than about 5%, or less than about 4%, or less than about 3%, or less than about 2%, or less than about 1%, or less than about 0.5% of the total amount of a gentisate form of Compound 1 when the pharmaceutically acceptable form is stored at 5 °C for at least 7 days. In other embodiments is provided a crystalline solid of a gentisate form of Compound 1, wherein the crystalline solid exhibits less than about 10% degradation of the total amount of a gentisate form of Compound 1 when the pharmaceutically acceptable form is stored at 5 °C for at least one month. In other embodiments is provided a crystalline solid of a gentisate form of Compound 1, wherein the crystalline solid exhibits less than about 9%, or less than about 8%, or less than about 7%, or less than about 6%, or less than about 5%, or less than about 4%, or less than about 3%, or less than about 2%, or less than about 1%, or less than about 0.5% of the total amount of a gentisate form of Compound 1 when the pharmaceutically acceptable form is stored at 5 °C for at least one month. In other embodiments is provided a crystalline solid of a gentisate form of Compound 1, wherein the crystalline solid exhibits less than about 10% degradation of the total amount of a gentisate form of Compound 1 when the pharmaceutically acceptable form is stored at 5 °C for at least 3 months. In other embodiments is provided a crystalline solid of a gentisate form of Compound 1, wherein the crystalline solid exhibits less than about 9%, or less than about 8%, or less than about 7%, or less than about 6%, or less than about 5%, or less than about 4%, or less than about 3%, or less than about 2%, or less than about 1%, or less than about 0.5% of the total amount of a gentisate form of Compound 1 when the pharmaceutically acceptable form is stored at 5 °C for at least 3 months.

In other embodiments is provided a crystalline solid of a gentisate form of Compound 1, wherein the crystalline solid exhibits less than about 10% degradation of the total amount of a gentisate form of Compound 1 when the pharmaceutically acceptable form is stored at 25 °C and 60% relative humidity for at least 7 days. In other embodiments is provided a crystalline solid of a gentisate form of Compound 1, wherein the crystalline solid exhibits less than about 9%, or less than about 8%, or less than about 7%, or less than about 6%, or less than about 5%, or less than about 4%, or less than about 3%, or less than about 2%, or less than about 1%, or less than about 0.5% of the total amount of a gentisate form of Compound 1 when the pharmaceutically acceptable form is stored at 25 °C and 60% relative humidity for at least 7 days. In other embodiments is provided a crystalline solid of a gentisate form of Compound 1, wherein the crystalline solid exhibits less than about 10% degradation of the total amount of a gentisate form of Compound 1 when the pharmaceutically acceptable form is stored at 25 °C and 60% relative humidity for at least one month. In other embodiments is provided a crystalline solid of a gentisate form of Compound 1, wherein the crystalline solid exhibits less than about 9%, or less than about 8%, or less than about 7%, or less than about 6%, or less than about 5%, or less than about 4%, or less than about 3%, or less than about 2%, or less than about 1%, or less than about 0.5% of the total amount of a gentisate form of Compound 1 when the pharmaceutically acceptable form is stored at 25 °C and 60% relative humidity for at least one month. In other embodiments is provided a crystalline solid of a gentisate form of Compound 1, wherein the crystalline solid exhibits less than about 10% degradation of the total amount of a gentisate form of Compound 1 when the pharmaceutically acceptable form is stored at 25 °C and 60% relative humidity for at least 3 months. In other embodiments is provided a crystalline solid of a gentisate form of Compound 1, wherein the crystalline solid exhibits less than about 9%, or less than about 8%, or less than about 7%, or less than about 6%, or less than about 5%, or less than about 4%, or less than about 3%, or less than about 2%, or less than about 1%, or less than about 0.5% of the total amount of a gentisate form of Compound 1 when the pharmaceutically acceptable form is stored at 25 °C and 60% relative humidity for at least 3 months.

In other embodiments is provided a crystalline solid of a gentisate form of Compound 1, wherein the crystalline solid exhibits less than about 10% degradation when the pharmaceutically acceptable form is stored at 40 °C and 75% relative humidity for at least 7 days. In other embodiments is provided a crystalline solid of a gentisate form of Compound 1, wherein the crystalline solid exhibits less than about 9%, or less than about 8%, or less than about 7%, or less than about 6%, or less than about 5%, or less than about 4%, or less than about 3%, or less than about 2%, or less than about 1%, or less than about 0.5% of the total amount of a gentisate form of Compound 1 when the pharmaceutically acceptable form is stored at 40 °C and 75% relative humidity for at least 7 days.

In other embodiments is provided a crystalline solid of a gentisate form of Compound 1, wherein the crystalline solid exhibits less than about 10% degradation of the total amount of a gentisate form of Compound 1 when the pharmaceutically acceptable form is stored at 60 °C for one or more day. In still further embodiments is provided a crystalline solid of a gentisate form of Compound 1, wherein the crystalline solid exhibits less than about 9%, or less than about 8%, or less than about 7%, or less than about 6%, or less than about 5%, or less than about 4%, or less than about 3%, or less than about 2%, or less than about 1%, or less than about 0.5% degradation of the total amount of a gentisate form of Compound 1 when the pharmaceutically acceptable form is stored at 60 °C for one or more days.

Also provided herein is a crystalline solid of a gentisate form of Compound 1, wherein the crystalline solid exhibits a peak in an x-ray powder diffraction (XRPD) pattern at 8.26±0.2° 2-theta. Further provided herein is a crystalline solid of a gentisate form of Compound 1, wherein the crystalline solid further exhibits a peak in an x-ray powder diffraction (XRPD) pattern at 26.43±0.2° 2-theta. In another embodiment is provided a crystalline solid of a gentisate form of Compound 1, wherein the crystalline solid further exhibits peaks in an x-ray powder diffraction (XRPD) pattern at 15.81±0.2° 2-theta and 15.40±0.2° 2-theta. In another embodiment is provided a crystalline solid of a gentisate form of Compound 1, wherein the crystalline solid further exhibits peaks in an x-ray powder diffraction (XRPD) pattern at 14.94±0.2° 2-theta and 20.44±0.2° 2-theta.

Also provided herein is a pharmaceutically acceptable form of Compound 1, wherein the form is a succinate form. In some embodiments, the succinate form comprises a succinate salt. In some embodiments, the succinate form comprises a succinate co-crystal. In further embodiments is provided a succinate form of Compound 1, wherein the molar ratio between Compound 1 and succinic acid is about 1:1. In further embodiments is provided a succinate form of Compound 1, wherein the molar ratio between Compound 1 and succinic acid is about 2:1. In further embodiments is provided a succinate form of Compound 1, wherein the molar ratio between Compound 1 and succinic acid is about 1:2. In other embodiments is provided such succinate form of Compound 1, wherein the succinate form is a hydrate. In some embodiments, the hydrate of the succinate form of Compound 1 is selected from a hemi-hydrate, a mono-hydrate, and a di-hydrate forms. In some embodiments, the hydrate is a hemi-hydrate. In some embodiments, the hydrate is a mono-hydrate. In some embodiments, the hydrate is a di-hydrate.

Further provided herein are pharmaceutical compositions, comprising an amount of a pharmaceutically acceptable form of Compound 1, and one or more pharmaceutically acceptable excipient, wherein the pharmaceutically acceptable form is selected from a gentisate form and a succinate form disclosed herein. In some embodiments, the gentisate form comprises a gentisate salt. In some embodiments, the gentisate form comprises a gentisate co-crystal. In some embodiment, the gentisate form comprises a salt and a co-crystal. In some embodiments, a succinate form comprises a succinate salt. In some embodiments, a succinate form comprises a succinate co-crystal. In some embodiments, the succinate form comprises a salt and a co-crystal.

Also provided herein are methods of inhibiting CD73 activity in a cell comprising contacting CD73 in the cell with an effective amount of a pharmaceutically acceptable form of Compound 1, wherein the pharmaceutically acceptable form is selected from a gentisate form and a succinate form disclosed herein.

Further provided herein are methods of treating cancer in a subject, comprising administering to the subject a therapeutically effective amount of a pharmaceutically acceptable form of Compound 1, wherein the pharmaceutically acceptable form is selected from a gentisate form and a succinate form disclosed herein. In other embodiments are provided methods of treating cancer in a subject, comprising administering to the subject a therapeutically effective amount of a pharmaceutical composition, the pharmaceutical composition comprising a pharmaceutically acceptable form of Compound 1, wherein the pharmaceutically acceptable form is selected from a gentisate form and a succinate form disclosed herein. In some embodiments, the cancer is lung cancer, melanoma, breast cancer, ovarian cancer, colorectal cancer, gastric cancer, gallbladder cancer, prostate cancer, renal cancer, multiple myeloma, or a lymphoma. In some embodiments, the cancer is lung cancer. In some embodiments, the cancer is melanoma. In some embodiments, the cancer is breast cancer. In some embodiments, the cancer is ovarian cancer. In some embodiments, the cancer is colorectal cancer. In some embodiments, the cancer is gastric cancer. In some embodiments, the cancer is gallbladder cancer. In some embodiments, the cancer is prostate cancer. In some embodiments, the prostate cancer is castration-resistant prostate, metastatic prostate cancer, metastatic castrate resistant prostate cancer, or castration-sensitive prostate cancer. In some embodiments, the cancer is renal cancer. In some embodiments, the cancer is multiple myeloma. In some embodiments, the multiple myeloma is light chain myeloma, non-secretory myeloma, solitary plasmacytoma, extramedullary plasmacytoma, monoclonal gammopathy of undetermined significance (MGUS), smoldering multiple myeloma (SMM), immunoglobulin D (IgD) myeloma, or immunoglobulin E (IgE) myeloma. In some embodiments, the cancer is a lymphoma.

Further provided herein are methods of treating cancer in a subject, comprising administering to the subject a therapeutically effective amount of a pharmaceutically acceptable form of Compound 1, wherein the pharmaceutically acceptable form is selected from a gentisate form and a succinate form disclosed herein, and wherein the cancer expresses CD73. In some embodiments, CD73 is upregulated in the cancer to be treated. In some embodiments, the cancer that expresses CD73 or in which CD73 is upregulated is lung cancer, melanoma, breast cancer, ovarian cancer, colorectal cancer, gastric cancer, gallbladder cancer, prostate cancer, renal cancer, multiple myeloma, or a lymphoma. In some embodiments, the cancer is lung cancer. In some embodiments, the cancer is melanoma. In some embodiments, the cancer is breast cancer. In some embodiments, the cancer is ovarian cancer. In some embodiments, the cancer is colorectal cancer. In some embodiments, the cancer is gastric cancer. In some embodiments, the cancer is gallbladder cancer. In some embodiments, the cancer is prostate cancer. In some embodiments, the prostate cancer is castration-resistant prostate, metastatic prostate cancer, metastatic castrate resistant prostate cancer, or castration-sensitive prostate cancer. In some embodiments, the cancer is renal cancer. In some embodiments, the cancer is multiple myeloma. In some embodiments, the multiple myeloma is light chain myeloma, non-secretory myeloma, solitary plasmacytoma, extramedullary plasmacytoma, monoclonal gammopathy of undetermined significance (MGUS), smoldering multiple myeloma (SMM), immunoglobulin D (IgD) myeloma, or immunoglobulin E (IgE) myeloma. In some embodiments, the cancer is a lymphoma.

Further provided herein are methods of treating cancer in a subject, comprising administering to the subject (a) a therapeutically effective amount of a pharmaceutically acceptable form of Compound 1, wherein the pharmaceutically acceptable form is selected from a gentisate form and a succinate form disclosed herein, and (b) one or more second therapeutic agents. In other embodiments are provided methods of treating cancer in a subject, comprising administering to the subject (a) a therapeutically effective amount of a pharmaceutical composition comprising a pharmaceutically acceptable form of Compound 1, wherein the pharmaceutically acceptable form is selected from a gentisate form and a succinate form disclosed herein, and (b) one or more second therapeutic agents. In some embodiments, the second therapeutic agent is a chemotherapeutic agent or an immunotherapy agent.

In further embodiments, the cancer in the subject is multiple myeloma and the second therapeutic agent is selected from chemotherapy, corticosteroids, immunomodulating agents, proteasome inhibitors, histone deacetylase (HDAC) inhibitors, monoclonal antibodies against CD38, monoclonal antibodies against SLAMF7, antibody-drug conjugates, and nuclear export inhibitors. In some embodiments, the second therapeutic agent is chemotherapy selected from cyclophosphamide, etoposide (VP-16), doxorubicin, liposomal doxorubicin, melphalan, melphalan flufenamide (melflufen), and bendamustine. In other embodiments, the second therapeutic agent is selected from corticosteroids including, but not limited to, dexamethasone and prednisone. In other embodiments, the second therapeutic agent is selected from immunomodulating agents including, but not limited to, thalidomide, lenalidomide, and pomalidomide. In further embodiments, the second therapeutic agent is selected from proteasome inhibitors including, but not limited to, bortezomib, carfilzomib, and ixazomib. In other embodiments, the second therapeutic agent is selected from histone deacetylase (HDAC) inhibitors including, but not limited to, Panobinostat. In still further embodiments, the second therapeutic agent is selected from monoclonal antibodies against CD38 including, but not limited to, daratumumab and isatuximab. In some embodiments, the second therapeutic agent is selected from antibodies against SLAMF7 including, but not limited to, elotuzumab. In some embodiments, the second therapeutic agent is selected from antibody-drug conjugates including, but not limited to, belantamab mafodotin. In some embodiments, the second therapeutic agent is selected from nuclear export inhibitors including, but not limited to, selinexor.

In other embodiments are provided methods of treating multiple myeloma in a subject, comprising administering to the subject (a) a therapeutically effective amount of a pharmaceutical composition comprising a pharmaceutically acceptable form of Compound 1, wherein the pharmaceutically acceptable form is selected from a gentisate form and a succinate form disclosed herein, and (b) one or more second therapeutic agents, wherein the one or more second therapeutic agents include, but are not limited to, the following combinations of agents: lenalidomide (or pomalidomide or thalidomide) and dexamethasone; carfilzomib (or ixazomib or bortezomib), lenalidomide, and dexamethasone; bortezomib (or carfilzomib), cyclophosphamide, and dexamethasone; elotuzumab (or daratumumab), lenalidomide, and dexamethasone; bortezomib, liposomal doxorubicin, and dexamethasone; panobinostat, bortezomib, and dexamethasone; elotuzumab, bortezomib, and dexamethasone; melphalan and prednisone (MP), with or without thalidomide or bortezomib; vincristine, doxorubicin, and dexamethasone (called VAD); dexamethasone, cyclophosphamide, etoposide, and cisplatin (called DCEP); dexamethasone, thalidomide, cisplatin, doxorubicin, cyclophosphamide, and etoposide (called DT-PACE), with or without bortezomib; and selinexor, bortezomib, and dexamethasone.

Further provided herein are methods of treating an infection in a subject, comprising administering to the subject a therapeutically effective amount of a pharmaceutically acceptable form of Compound 1, wherein the pharmaceutically acceptable form is selected from a gentisate form and a succinate form disclosed herein. In some embodiments, the infection is a viral infection. In other embodiments, the infection is a parasitic infection.

Further provided herein are methods of treating a neurodegenerative disease in a subject, comprising administering to the subject a therapeutically effective amount of a pharmaceutically acceptable form of Compound 1, wherein the pharmaceutically acceptable form is selected from a gentisate form and a succinate form disclosed herein. Also provided herein are methods of treating a neurodegenerative disease in a subject, comprising administering to the subject a therapeutically effective amount of a pharmaceutical composition, wherein the pharmaceutical comprises a pharmaceutically acceptable form of Compound 1, and wherein the pharmaceutically acceptable form is selected from a gentisate form and a succinate form disclosed herein. In some embodiments, the neurodegenerative disease is Alzheimer's disease, Parkinson's disease, Huntington's disease, schizophrenia, or autism.

Further provided herein are compositions for use in treating cancer in a subject, the compositions comprising a form of Compound 1. In some embodiments, the compositions comprise a succinate form of Compound 1. In some embodiments, the compositions comprise a gentisate form of Compound 1. In some embodiments, the compositions comprise a gentisate form of Compound 1, wherein the form is crystalline. In some embodiments, the compositions comprise a gentisate form of Compound 1, wherein the form is in crystalline solid that exhibits a peak in an x-ray powder diffraction (XRPD) pattern at 9.3±0.2° 2-theta. In still further embodiments, the crystalline solid exhibits a further peak in an x-ray powder diffraction (XRPD) pattern at 7.1±0.2° 2-theta. In still further embodiments, the crystalline solid exhibits a further peak in an x-ray powder diffraction (XRPD) pattern at 20.1±0.2° 2-theta. In still further embodiments, the crystalline solid exhibits a further peak in an x-ray powder diffraction (XRPD) pattern at 19.0±0.2° 2-theta. In still further embodiments, the crystalline solid exhibits a further peak in an x-ray powder diffraction (XRPD) pattern at 26.2±0.2° 2-theta. In still further embodiments, the crystalline solid exhibits further peaks in an x-ray powder diffraction (XRPD) pattern at 4.8±0.2° 2-theta, 10.5±0.2° 2-theta, and 14.8±0.2° 2-theta. In some embodiments, the cancer is lung cancer, melanoma, breast cancer, ovarian cancer, colorectal cancer, gastric cancer, gallbladder cancer, prostate cancer, renal cancer, multiple myeloma, or a lymphoma. In some embodiments, the cancer is lung cancer. In some embodiments, the cancer is melanoma. In some embodiments, the cancer is breast cancer. In some embodiments, the cancer is ovarian cancer. In some embodiments, the cancer is colorectal cancer. In some embodiments, the cancer is gastric cancer. In some embodiments, the cancer is gallbladder cancer. In some embodiments, the cancer is prostate cancer. In some embodiments, the prostate cancer is castration-resistant prostate, metastatic prostate cancer, metastatic castrate resistant prostate cancer, or castration-sensitive prostate cancer. In some embodiments, the cancer is renal cancer. In some embodiments, the cancer is multiple myeloma. In some embodiments, the multiple myeloma is light chain myeloma, non-secretory myeloma, solitary plasmacytoma, extramedullary plasmacytoma, monoclonal gammopathy of undetermined significance (MGUS), smoldering multiple myeloma (SMM), immunoglobulin D (IgD) myeloma, or immunoglobulin E (IgE) myeloma. In some embodiments, the cancer is a lymphoma. In some embodiments, the cancer expresses CD73. In some embodiments, CD73 is upregulated in the cancer to be treated. In some embodiments, the cancer that expresses CD73 or in which CD73 is upregulated is lung cancer, melanoma, breast cancer, ovarian cancer, colorectal cancer, gastric cancer, gallbladder cancer, prostate cancer, renal cancer, multiple myeloma, or a lymphoma. In some embodiments, the cancer is lung cancer. In some embodiments, the cancer is melanoma. In some embodiments, the cancer is breast cancer. In some embodiments, the cancer is ovarian cancer. In some embodiments, the cancer is colorectal cancer. In some embodiments, the cancer is gastric cancer. In some embodiments, the cancer is gallbladder cancer. In some embodiments, the cancer is prostate cancer. In some embodiments, the prostate cancer is castration-resistant prostate, metastatic prostate cancer, metastatic castrate resistant prostate cancer, or castration-sensitive prostate cancer. In some embodiments, the cancer is renal cancer. In some embodiments, the cancer is multiple myeloma. In some embodiments, the multiple myeloma is light chain myeloma, non-secretory myeloma, solitary plasmacytoma, extramedullary plasmacytoma, monoclonal gammopathy of undetermined significance (MGUS), smoldering multiple myeloma (SMM), immunoglobulin D (IgD) myeloma, or immunoglobulin E (IgE) myeloma. In some embodiments, the cancer is a lymphoma.

Also provided herein are pharmaceutical compositions comprising an amount of a pharmaceutically acceptable form of Compound 1, wherein the pharmaceutically acceptable form is selected from a gentisate form and a succinate form, and one or more pharmaceutically acceptable excipients. In some embodiments, a gentisate form comprises a gentisate salt. In some embodiments, a gentisate form comprises a gentisate co-crystal. In some embodiments, the gentisate form comprises a salt and a co-crystal. In some embodiments, a succinate form comprises a succinate salt. In some embodiments, a succinate form comprises a succinate co-crystal. In some embodiments, the succinate form comprises a salt and a co-crystal. Further provided herein are such pharmaceutical compositions, wherein the one or more pharmaceutically acceptable excipients comprises one or more diluents, binders, disintegrants, lubricants, antiadhesives, glidants, coloring agents, flavors, sweeteners, coating agents, plasticizers wetting agents, buffers, or adsorbents.

Among the one or more diluents that may be used are lactose, mannitol, xylitol, microcrystalline cellulose, dibasic calcium phosphate and starch. In some embodiments, the one or more diluents comprises from about 1% to about 80%, or from about 10% to about 80%, or from about 10% to about 70%, or from about 15% to about 80%, or from about 20% to about 80%, or from about 15% to about 75%, or from about 20% to about 75%, or from about 25% to about 75%, or from about 50% to about 80%, or from about 50% to about 75%, or from about 60% to about 80%, or from about 60% to 75% of the total weight of the pharmaceutical compositions. In some embodiments, the diluent is lactose. In some embodiments, the diluent is mannitol. In some embodiments, the diluent is xylitol. In some embodiments, the diluent is microcrystalline cellulose. In some embodiments, the diluent is dibasic calcium phosphate. In some embodiments, the diluent is starch.

Also provided herein are such pharmaceutical compositions, wherein the one or more pharmaceutically acceptable excipients comprises one or more binders, wherein the one or more binders comprises from about 1% to about 80%, or from about 10% to about 80%, or from about 10% to about 70%, or from about 15% to about 80%, or from about 20% to about 80%, or from about 15% to about 75%, or from about 20% to about 75%, or from about 25% to about 75%, or from about 50% to about 80%, or from about 50% to about 75%, or from about 60% to about 80%, or from about 60% to 75% of the total weight of the pharmaceutical compositions. In some embodiments, the one or more binders is selected from selected from methyl cellulose, microcrystalline cellulose, starch, and gums such as guar gum, and tragacanth, or a mixture thereof.

Also provided herein are such pharmaceutical compositions, wherein the one or more pharmaceutically acceptable excipients comprises one or more disintegrants, and wherein the one or more disintegrants comprises from about 0.1% to about 10%, or from about 0.1% to about 5%, or from about 0.1% to about 4%, or from about 0.1% to about 3%, or from about 0.1% to about 2%, or from about 0.1% to about 1%, or from about 0.1% to about 0.75%, or from about 0.2% to about 1%, or from about 0.3% to about 1%, or from about 0.4% to about 1%, or from about 0.2% to about 0.8%, or from about 0.3% to about 0.75%, or from about 0.3% to about 0.7%, or from about 0.3% to about 0.6% by weight of the total weight of the pharmaceutical compositions. In some embodiments the one or more disintegrants is selected from starch, sodium starch glycolate, sodium alginate, carboxymethylcellulose sodium, methyl cellulose, croscarmellose sodium and crospovidone, or mixtures thereof. In some embodiments, the disintegrant is starch. In some embodiments, the disintegrant is sodium starch glycolate. In some embodiments, the disintegrant is sodium alginate. In some embodiments, the disintegrant is carboxymethylcellulose sodium. In some embodiments, the disintegrant is methyl cellulose. In some embodiments, the disintegrant is croscarmellose sodium. In some embodiments, the disintegrant is crospovidone.

Also provided herein are such pharmaceutical compositions, wherein the one or more pharmaceutically acceptable excipients comprises one or more lubricants, and wherein the one or more lubricants comprises from about 0.1% to about 10%, or from about 0.1% to about 5%, or from about 0.10% to about 4%, or from about 0.1% to about 3%, or from about 0.1% to about 2%, or from about 0.1% to about 10%, or from about 0.1% to about 0.75%, or from about 0.2% to about 10%, or from about 0.3% to about 1%, or from about 0.4% to about 1%, or from about 0.2% to about 0.8%, or from about 0.3% to about 0.75%, or from about 0.3% to about 0.7%, or from about 0.3% to about 0.6% by weight of the total weight of the pharmaceutical compositions. In a further embodiment, the one or more lubricants is selected from magnesium stearate, calcium stearate, sodium stearyl fumarate, and stearic acid, or mixtures thereof. In some embodiments, the lubricant is magnesium stearate. In some embodiments, the lubricant is calcium stearate. In some embodiments, the lubricant is sodium stearyl fumarate. In some embodiments, the lubricant is stearic acid.

In some embodiments, the pharmaceutical compositions disclosed herein may comprise additional excipients including, but not limited to, buffering agents, glidants, preservatives, and coloring agents. Additional excipients such as bulking agents, tonicity agents, and chelating agents are also within the scope of the embodiments.

Non-limiting examples of buffering agents include, but are not limited to, sodium bicarbonate, potassium bicarbonate, magnesium hydroxide, magnesium lactate, magnesium glucomate, aluminum hydroxide, aluminum hydroxide/sodium bicarbonate co precipitate, a mixture of an amino acid and a buffer, a mixture of aluminum glycinate and a buffer, a mixture of an acid salt of an amino acid and a buffer, and a mixture of an alkali salt of an amino acid and a buffer. Additional buffering agents include sodium citrate, sodium tartarate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate, and other calcium salts.

In some embodiments, the pharmaceutical compositions disclosed herein may comprise a glidant. Suitable glidants include, but are not limited to, calcium phosphate tribasic, calcium silicate, cellulose, colloidal silicon dioxide, magnesium silicate, magnesium trisilicate, silicon dioxide, starch, talc, and the like. In some embodiments, the glidant is calcium phosphate tribasic. In some embodiments, the glidant is calcium silicate. In some embodiments, the glidant is cellulose. In some embodiments, the glidant is colloidal silicon dioxide. In some embodiments, the glidant is magnesium silicate. In some embodiments, the glidant is magnesium trisilicate. In some embodiments, the glidant is silicon dioxide. In some embodiments, the glidant is starch. In some embodiments, the glidant is talc.

In some embodiments, the pharmaceutical compositions disclosed herein may comprise a preservative. Preservatives include anti-microbials, anti-oxidants, and agents that enhance sterility. Exemplary preservatives include ascorbic acid, ascorbyl palmitate, BHA, BHT, citric acid, erythorbic acid, fumaric acid, malic acid, propyl gallate, sodium ascorbate, sodium bisulfate, sodium metabisulfite, sodium sulfite, parabens (methyl-, ethyl-, butyl-), benzoic acid, potassium sorbate, vanillin, and the like.

In some embodiments, the pharmaceutical compositions disclosed herein may comprise a coloring agent for identity and/or aesthetic purposes of the resultant liquid form. Suitable coloring agents illustratively include FD&C Red No. 3, FD&C Red No. 20, FD&C Red No. 40, FD&C Yellow No. 6, FD&C Blue No. 2, D&C Green No. 5, D&C Orange No. 5, caramel, ferric oxide, and mixtures thereof.

Additional excipients are contemplated in the pharmaceutical compositions disclosed herein. These additional excipients are selected based on function and compatibility with the pharmaceutical compositions described herein and may be found, for example in Remington: The Science and Practice of Pharmacy, Nineteenth Ed (Easton, PA: Mack Publishing Company, 1995); Hoover, John E., Remington's Pharmaceutical Sciences, (Easton, PA: Mack Publishing Co 1975); Liberman, H. A. and Lachman, L., Eds., Pharmaceutical Dosage Forms (New York, NY: Marcel Decker 1980); and Pharmaceutical Dosage Forms and Drug Delivery Systems, Seventh Ed (Lippincott Williams & Wilkins 1999), herein incorporated by reference in their entirety.

The pharmaceutical compositions disclosed herein may be in a form suitable for oral dosage to a subject in need. Suitable oral dosage forms include, for example, tablets, pills, sachets, or capsules of hard or soft gelatin, methylcellulose or of another suitable material easily dissolved in the digestive tract. Oral administration of a solid dose form may be, for example, presented in discrete units, such as hard or soft capsules, pills, cachets, lozenges, or tablets, each containing a predetermined amount of a form of Compound 1 as disclosed herein and one or more pharmaceutically acceptable excipients. In another embodiment, the oral administration may be in a powder or granule form. In another embodiment, the oral dose form is sub-lingual, such as, for example, a lozenge. Capsules or tablets may contain a controlled-release formulation. In the case of capsules, tablets, and pills, the dosage forms also may comprise buffering agents or may be prepared with enteric coatings. In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

Also provided herein are tablets comprising a pharmaceutically acceptable form of Compound 1, microcrystalline cellulose, colloidal silica dioxide, sodium stearyl fumarate, crospovidone, and magnesium stearate. In some embodiments, the pharmaceutically acceptable form of Compound 1 is selected from a gentisate form and a succinate form. In some embodiments, the pharmaceutically acceptable form of Compound 1 is a gentisate form. In some embodiments, the gentisate form comprises a salt. In some embodiments, the gentisate form comprises a co-crystal. In some embodiments, the gentisate form comprises a salt and a co-crystal. In some embodiments, the pharmaceutically acceptable form of Compound 1 is a succinate form. In some embodiments, the succinate form comprises a salt. In some embodiments, the succinate form comprises a co-crystal. In some embodiments, the succinate form comprises a salt and a co-crystal. In further embodiments, the tablets disclosed herein are as set forth in Table 1.

TABLE 1

| | Amount per | Weight percent |
|---|---|---|
| Material | tablet (mg) | of tablet |
| Compound 1 gentisate form | 261.438 | 38.45% |
| Microcrystalline | 377.162 | 55.46% |
| cellulose PH | | |
| Colloidal silica dioxide | 7.000 | 1.03% |
| Sodium stearyl fumarate | 7.000 | 1.03% |
| Crospovidone | 20.400 | 3.00% |
| Magnesium stearate | 7.000 | 1.03% |
| Total | 680 | 100% |

In another embodiment, the pharmaceutical compositions disclosed herein may comprise a parenteral dose form. “Parenteral administration” includes, for example, subcutaneous injections, intravenous injections, intraperitoneal injections, intramuscular injections, intrasternal injections, and infusion. Injectable preparations (e.g., sterile injectable aqueous or oleaginous suspensions) may be formulated according to the known art using suitable dispersing, wetting agents, and/or suspending agents.

In another embodiment, the pharmaceutical compositions disclosed herein may comprise a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound that enhances absorption or penetration of the active ingredient through the skin or other affected areas. When the compositions disclosed herein are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J. Pharm. Sci., 88(10), 955-958, by Finnin and Morgan (October 1999).

For intranasal administration or administration by inhalation, the pharmaceutical compositions disclosed herein are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

Other carrier materials and modes of administration known in the pharmaceutical art may also be used. Pharmaceutical compositions disclosed herein may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3.sup.rd Ed.), American Pharmaceutical Association, Washington, 1999.

The dose of the composition comprising a form of Compound 1 as described herein may differ, depending upon the patient's (e.g., human) condition, that is, stage of the disease, general health status, age, and other factors. Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient. Oral doses typically range from about 1.0 mg to about 1000 mg, one to four times, or more, per day.

Compound 1 may be prepared by methods known to those having ordinary skill in the art, including, but not limited to, the methods set forth in Example 1.

Among the abbreviations used in the Examples are: rt (room temperature), min (minute(s)), h (hour(s)), MeCN (acetonitrile), DMF (N,N-dimethylformamide), THF (tetrahydrofuran), MeOH (methanol), sat (saturated), and TsOH (toluene sulfonic acid).

### EXAMPLES

### Example 1. Preparation of ((S)-1-((2H-tetrazol-5-yl)methoxy)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-hydroxypropan-2-yl)phosphonic acid

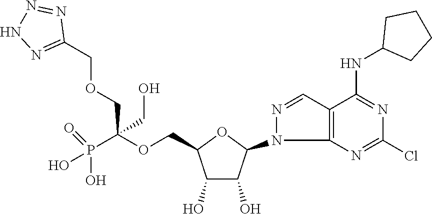 [SMILES: O=P(O)(O)[C@@](CO)(COCc1nn[nH]n1)OC[C@H]1O[C@@H](n2ncc3c(NC4CCCC4)nc(Cl)nc32)[C@H](O)[C@@H]1O]

### Step A. (2R,3R,4R,5R)-2-(Acetoxymethyl)-5-(4,6-dichloro-1H-pyrazolo[3,4-d]pyrimidin-1-yl)tetrahydrofuran-3,4-diyl diacetate (1a)

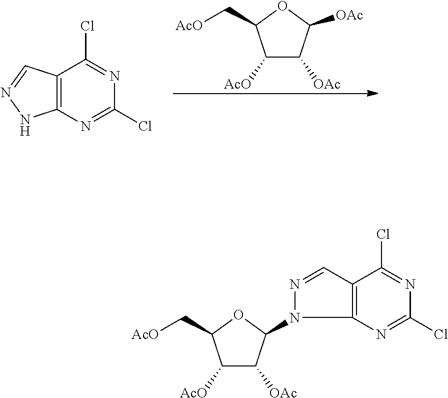 [SMILES: Clc1nc(Cl)c2cn[nH]c2n1 ; CC(=O)OC[C@H]1O[C@@H](OC(C)=O)[C@H](OC(C)=O)[C@@H]1OC(C)=O ; CC(=O)OC[C@H]1O[C@@H](n2ncc3c(Cl)nc(Cl)nc32)[C@H](OC(C)=O)[C@@H]1OC(C)=O]

β-D-Ribofuranose 1,2,3,5-tetraacetate (5.73 g, 17.99 mmol) was heated at 90 °C for 10 min, 4,6-dichloro-1H-pyrazolo[3,4-d]pyrimidine (1.5 g, 17.99 mmol) and SnCl₄ (60 mg) was added successively. After the mixture was heated at 130 °C under reduced pressure for 15 min, it was cooled to rt, diluted with water, and extracted with DCM. The combined organics were washed with water, brine, dried and concentrated. The residue was purified by column chromatography (petroleum ether/ethyl acetate from 10:1 to 5:1) to give the title compound (1a) (2.4 g, 68%) as a yellow solid.

### Step B. (2R,3R,4R,5R)-2-(Acetoxymethyl)-5-(6-chloro-4-(cyclopentylamino)-1H-pyrazolo[3,4-d]pyrimidin-1-yl)tetrahydrofuran-3,4-diyl diacetate (1b)

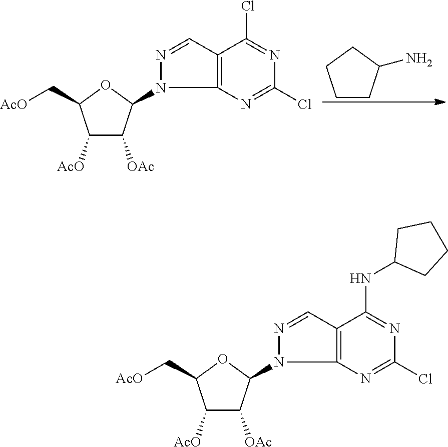

To an oven-dried flask was added 1a (5.2 g, 11.63 mmol) followed by ethanol (53.24 mL). To this solution was added triethylamine (2.43 mL, 17.44 mmol) followed by cyclopentylamine (1.38 mL, 13.95 mmol). After the mixture was stirred and heated at 50 °C for 15 min, it was cooled to rt, concentrated, and purified by column chromatography (20 to 45% ethyl acetate/hexanes, a gradient elution) to provide the title compound (1b) (5.02 g, 87%) as a white solid. m/z (ESI, +ve ion)=496.1 [M+H]⁺.

### Step C. (2R,3R,4R,5R)-2-(Acetoxymethyl)-5-(4-((tert-butoxycarbonyl)(cyclopentyl)amino)-6-chloro-1H-pyrazolo[3,4-d]pyrimidin-1-yl)tetrahydrofuran-3,4-diyl diacetate (1c)

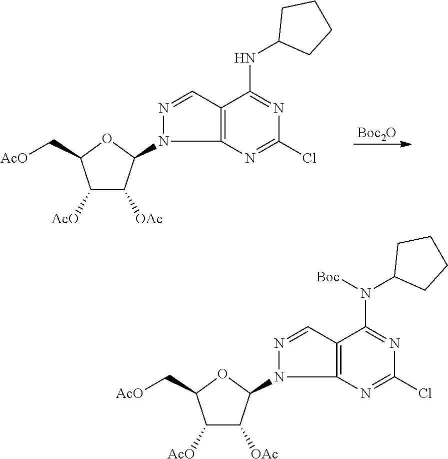 [SMILES: "Boc2O" ; CC(=O)OC[C@H]1O[C@@H](n2ncc3c(NC4CCCC4)nc(Cl)nc32)[C@H](OC(C)=O)[C@@H]1OC(C)=O ; CC(=O)OC[C@H]1O[C@@H](n2ncc3c(N(C(=O)OC(C)(C)C)C4CCCC4)nc(Cl)nc32)[C@H](OC(C)=O)[C@@H]1OC(C)=O]

To a solution of 1b (12.6 g, 25.4 mmol) in MeCN (120 mL) was added triethylamine (5.14 g, 50.9 mmol) followed by di-tert-butyl dicarbonate (44.35 g, 203.6 mmol) and 4-dimethylaminopyridine (0.31 g, 2.54 mmol). After the mixture was allowed to stir overnight, it was concentrated and partitioned between EtOAc (50 mL) and sat. NaHCO₃. The organic layer was washed with brine, dried with Na₂SO₄, filtered, and concentrated. The residue was purified by column chromatography (petroleum ether/ethyl acetate: 8:1) to provide the title compound (1c) (10.56 g, 70% yield) as a yellow solid. m/z (ESI, +ve ion)=596.72 [M+H]⁺.

### Step D. tert-Butyl (6-chloro-1-((2R,3R,4S,5R)-3,4-dihydroxy-5-(hydroxymethyl)tetrahydrofuran-2-yl)-1H-pyrazolo[3,4-d]pyrimidin-4-yl)(cyclopentyl)carbamate (1d)

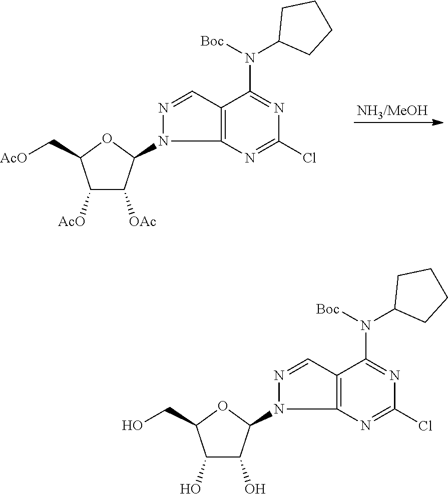

To an oven-dried flask was added 1c (10.56 g, 17.78 mmol), followed by ammonia (5.0 M, 140 mL) in methanol. The mixture was stirred overnight and then concentrated. The crude oil was purified by column chromatography to afford the title compound (1d) (7.39 g, 89% yield) as a yellow solid. m/z (ESI, +ve ion)=470.3 [M+H]⁺.

### Step E. tert-Butyl (6-chloro-1-((3aR,4R,6R,6aR)-6-(hydroxymethyl)-2,2-dimethyltetrahydrofuro[3,4-d][1,3]dioxol-4-yl)-1H-pyrazolo[3,4-d]pyrimidin-4-yl)(cyclopentyl)carbamate (1e)

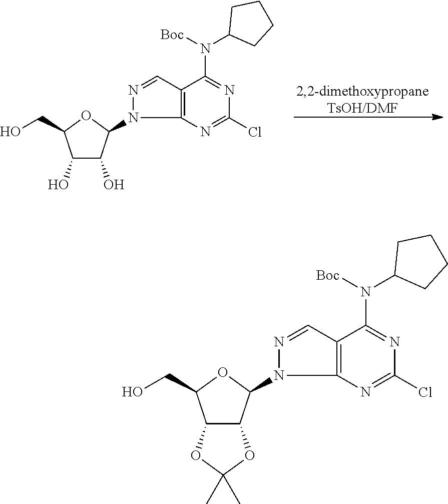 [SMILES: CC(C)(C)OC(=O)N(c1nc(Cl)nc2c1cnn2[C@@H]1O[C@H](CO)[C@@H](O)[C@H]1O)C1CCCC1 ; "2,2-dimethoxypropaneTsOH/DMF" ; CC(C)(C)OC(=O)N(c1nc(Cl)nc2c1cnn2[C@@H]1O[C@H](CO)[C@H]2OC(C)(C)O[C@H]21)C1CCCC1]

To a solution of 1d (7.39 g, 15.75 mmol) and 2,2-dimethoxypropane (4.92 g, 47.27 mmol) in DMF (75 mL) was added TsOH·H₂O (0.6 g, 3.15 mmol). After the mixture was stirred at 70 °C for 1 h, it was cooled down and quenched with sat. NaHCO₃ (100 mL). The mixture was extracted with EtOAc (50 mL) and the combined organic layers were washed with brine, dried with Na₂SO₄, filtered, and concentrated. The crude oil was purified by column chromatography (petroleum ether/ethyl acetate: 8:1) to afford the title compound (1e) (5.5 g, 68% yield) as a yellow solid. m/z (ESI, +ve ion)=510.4 [M+H]⁺.

### Step F. Ethyl 2-(((3aR,4R,6R,6aR)-6-(4-((tert-butoxycarbonyl)(cyclopentyl)amino)-6-chloro-1H-pyrazolo[3,4-d]pyrimidin-1-yl)-2,2-dimethyltetrahydrofuro[3,4-d][1,3]dioxol-4-yl)methoxy)-2-(diethoxyphosphoryl)acetate (1f)

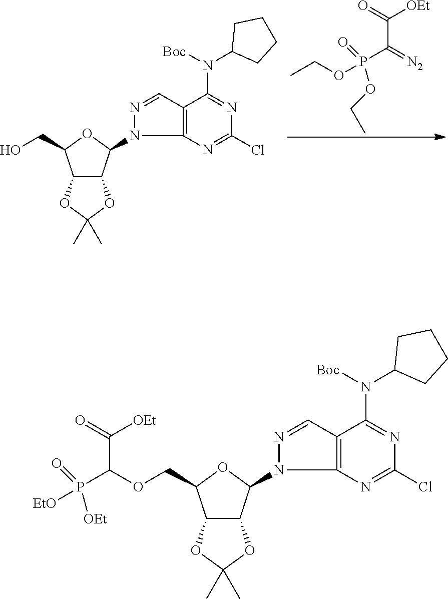

To a solution of ethyl 2-diazo-2-(diethoxyphosphoryl)acetate (13.5 g, 54.13 mmol) and 1e (5.5 g, 10.83 mmol) in toluene (80 mL) was added Rh₂(OAc)₄ (0.96 g, 2.17 mmol) under N₂. After the mixture was stirred at 95 °C overnight, it was concentrated and purified by column chromatography (petroleum ether/ethyl acetate: 5:1) to afford the title compound (1f) (6 g, 76% yield) as a yellow oil. m/z (ESI, +ve ion)=732.2 [M+H]⁺.

### Step G. Ethyl 2-[[(3aR,4R,6R,6aR)-4-[4-[tert-butoxycarbonyl(cyclopentyl)amino]-6-chloro-pyrazolo[3,4-d]pyrimidin-1-yl]-2,2-dimethyl-3a,4,6,6a-tetrahydrofuro[3,4-d][1,3]dioxol-6-yl]methoxy]-2-diethoxyphosphoryl-3-(2-trimethylsilylethoxy)propanoate (1g)

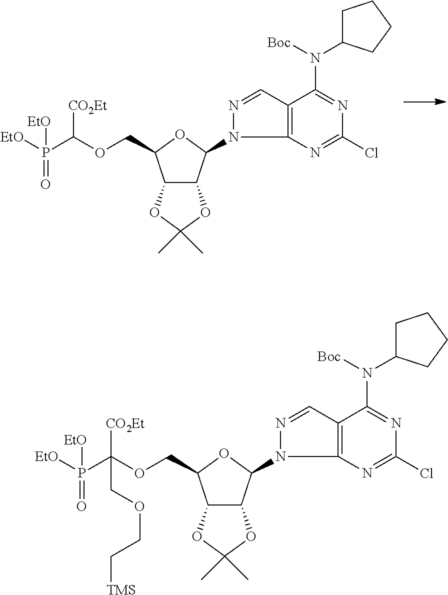 [SMILES: CCOC(=O)C(OC[C@H]1O[C@@H](n2ncc3c(N(C(=O)OC(C)(C)C)C4CCCC4)nc(Cl)nc32)[C@@H]2OC(C)(C)O[C@@H]21)P(=O)(OCC)OCC ; CCOC(=O)C(COCC[Si](C)(C)C)(OC[C@H]1O[C@@H](n2ncc3c(N(C(=O)OC(C)(C)C)C4CCCC4)nc(Cl)nc32)[C@@H]2OC(C)(C)O[C@@H]21)P(=O)(OCC)OCC]

To a solution of the compound 1f (1.2 g, 1.64 mmol) in THF (33 mL) was added dropwise sodium bis(trimethylsilyl)amide (1.0 M in THF, 2.13 mL, 2.13 mmol) at −15 °C. After stirring at −15 °C for 25 min, tetra-n-butylammonium iodide (303 mg, 0.820 mmol) was added, immediately followed by the dropwise addition of 2-(chloromethoxy)ethyl](trimethyl)silane (0.863 mL, 4.92 mmol) to the solution. The mixture was stirred at the same temperature for 1 h and then quenched with sat. aq. NH₄Cl. The solution was diluted with EtOAc and water, extracted with EtOAc. The combined organic layers were washed (brine), dried (Na₂SO₄), and concentrated under reduced pressure. Purification of the residue by silica gel column chromatography (5-30% acetone/hexanes, a gradient elution) provided the title compound (1g) (1.03 g, 73%) as a light-yellow oil. m/z (ESI, +ve ion)=862.3 [M+H]⁺.

### Step H. tert-Butyl N-[1-[(3aR,4R,6R,6aR)-6-[[1-diethoxyphosphoryl-1-(hydroxymethyl)-2-(2-trimethylsilylethoxy)ethoxy]methyl]-2,2-dimethyl-3a,4,6,6a-tetrahydrofuro[3,4-d][1,3]dioxol-4-yl]-6-chloro-pyrazolo[3,4-d]pyrimidin-4-yl]-N-cyclopentyl-carbamate (1h)

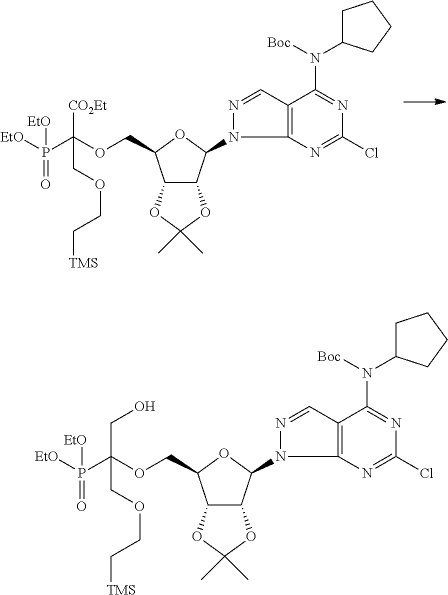 [SMILES: CCOC(=O)C(COCC[Si](C)(C)C)(OC[C@H]1O[C@@H](n2ncc3c(N(C(=O)OC(C)(C)C)C4CCCC4)nc(Cl)nc32)[C@@H]2OC(C)(C)O[C@@H]21)P(=O)(OCC)OCC ; CCOP(=O)(OCC)C(CO)(COCC[Si](C)(C)C)OC[C@H]1O[C@@H](n2ncc3c(N(C(=O)OC(C)(C)C)C4CCCC4)nc(Cl)nc32)[C@@H]2OC(C)(C)O[C@@H]21]

To a stirred solution of ethyl 2-[[(3aR,4R,6R,6aR)-4-[4-[tert-butoxycarbonyl(cyclopentyl)amino]-6-chloro-pyrazolo[3,4-d]pyrimidin-1-yl]-2,2-dimethyl-3a,4,6,6a-tetrahydrofuro[3,4-d][1,3]dioxol-6-yl]methoxy]-2-diethoxyphosphoryl-3-(2-trimethylsilylethoxy)propanoate (1g) (1.03 g, 1.19 mmol) in EtOH (18 mL) was added calcium dichloride (596 g, 5.37 mmol), followed by addition of sodium borohydride (203 mg, 5.37 mmol) in a single portion at 0 °C. The mixture was allowed to warm to rt and stirred for 3 h. The mixture was then cooled back to 0 °C, and the mixture was quenched with aq. IN HCl, diluted with EtOAc and water. The solution was extracted (EtOAc) and the combined organic layers were washed (brine), dried (Na₂SO₄), and concentrated under reduced pressure. Purification of the residue by silica gel column chromatography (1-5% MeOH/DCM, a gradient elution) provided the title compound (1h) (755 mg, 77%) as a white foamy solid. m/z (ESI, +ve ion)=820.3 [M+H]⁺.

### Step I. tert-butyl (6-chloro-1-((3aR,4R,6R,6aR)-6-(((2-(diethoxyphosphoryl)-1-(2-(trimethylsilyl)ethoxy)-3-((2-((2-(trimethylsilyl)ethoxy)methyl)-2H-tetrazol-5-yl)methoxy)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (li)

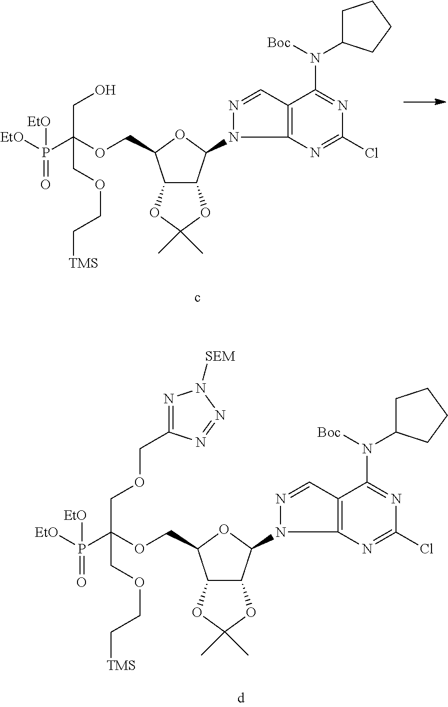 [SMILES: CCOP(=O)(OCC)C(CO)(COCC[Si](C)(C)C)OC[C@H]1O[C@@H](n2ncc3c(N(C(=O)OC(C)(C)C)C4CCCC4)nc(Cl)nc32)[C@@H]2OC(C)(C)O[C@@H]21 ; CCOP(=O)(OCC)C(COCC[Si](C)(C)C)(COCc1nnn(COCC[Si](C)(C)C)n1)OC[C@H]1O[C@@H](n2ncc3c(N(C(=O)OC(C)(C)C)C4CCCC4)nc(Cl)nc32)[C@@H]2OC(C)(C)O[C@@H]21]

To a solution of tert-Butyl N-[1-[(3aR,4R,6R,6aR)-6-[[1-diethoxyphosphoryl-1-(hydroxymethyl)-2-(2-trimethylsilylethoxy)ethoxy]methyl]-2,2-dimethyl-3a,4,6,6a-tetrahydrofuro[3,4-d][1,3]dioxol-4-yl]-6-chloro-pyrazolo[3,4-d]pyrimidin-4-yl]-N-cyclopentyl-carbamate (1h) (632 mg, 0.770 mmol) and 5-(bromomethyl)-2-((2-(trimethylsilyl)ethoxy)methyl)-2H-tetrazole (1.13 g, 3.85 mmol) and in DMF (5.0 mL) at 0 °C was added NaH (60% mineral oil, 77.0 mg, 1.93 mmol) in one portion. After the mixture was stirred at 0 °C for 30 min, the mixture was quenched by sat. aq. NH₄Cl, diluted with EtOAc and water. The solution was extracted (EtOAc) and the combined organic layers were washed (brine), dried (Na₂SO₄), and concentrated under reduced pressure. The resulting residue was purified by flash chromatography (5-30% acetone/hexanes, a gradient elution) to afford the title compound (ii) (697 mg, 88%) as a light-yellow gum.

### Step J. tert-butyl (6-chloro-1-((3aR,4R,6R,6aR)-6-((((R)-2-(diethoxyphosphoryl)-1-(2-(trimethylsilyl)ethoxy)-3-((2-((2-(trimethylsilyl)ethoxy)methyl)-2H-tetrazol-5-yl)methoxy)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (1j)

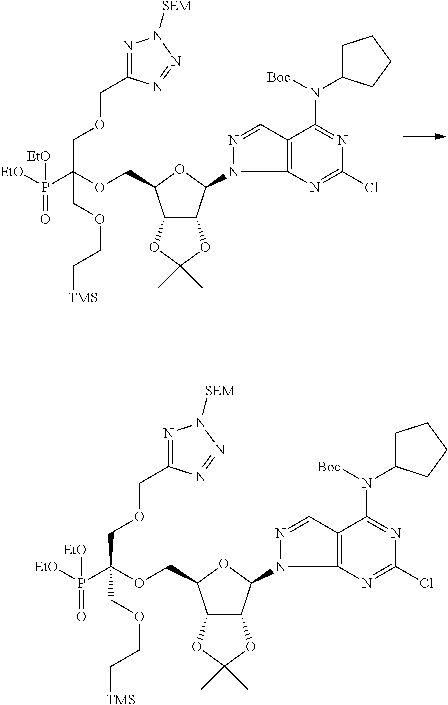

The diastereomers from Step I were separated by chiral chromatography (CHIRALPAK, AD-H, 21×250 mm, 5 μm, 5% IPA/hexanes, an isocratic elution, a flow rate of 20 mL/min), and the second eluted isomer was identified as the title compound (1j) and was collected.

### Step K. diethyl ((S)-1-((2H-tetrazol-5-yl)methoxy)-2-(((3aR,4R,6R,6aR)-6-(6-chloro-4-(cyclopentylamino)-1H-pyrazolo[3,4-d]pyrimidin-1-yl)-2,2-dimethyltetrahydrofuro[3,4-d][1,3]dioxol-4-yl)methoxy)-3-hydroxypropan-2-yl)phosphonate (1k)

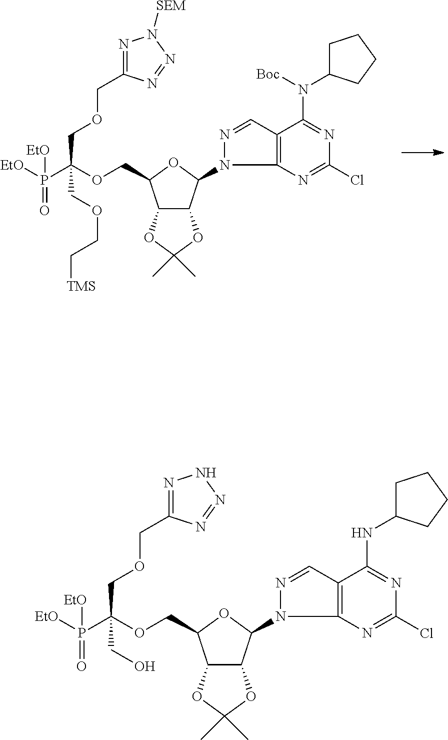

To a solution of tert-butyl (6-chloro-1-((3aR,4R,6R,6aR)-6-((((R)-2-(diethoxyphosphoryl)-1-(2-(trimethylsilyl)ethoxy)-3-((2-((2-(trimethylsilyl)ethoxy)methyl)-2H-tetrazol-5-yl)methoxy)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (1j) (325 mg, 0.315 mmol) in DCM (16 mL) was added dropwise boron trifluoride diethyl etherate (0.233 mL, 1.89 mmol) at 0 °C. The reaction was allowed to warm to rt. After stirring at rt for 3.5 h, the reaction was quenched with triethylamine (3.6 mL) and the resulting mixture was stirred at rt for 10 min. sat. aq. NaHCO₃ (7.2 mL) was added to the mixture and the solution was diluted with DCM and water. The solution was extracted (DCM) and the combined organic layers were washed (brine), dried (Na₂SO₄), and concentrated under reduced pressure. The resulting residue was purified by flash chromatography (0-20% MeOH/DCM, a gradient elution) to afford the title compound (1k) (189 mg, 86%) as an off-white foamy solid. m/z (ESI, +ve ion)=702.3 [M+H]⁺.

### Step L. ((S)-1-((2H-tetrazol-5-yl)methoxy)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-hydroxypropan-2-yl)phosphonic acid (1)

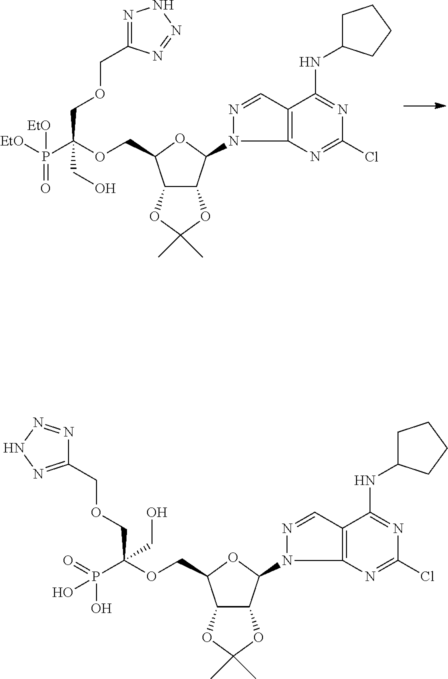

To a solution of diethyl ((S)-1-((2H-tetrazol-5-yl)methoxy)-2-(((3aR,4R,6R,6aR)-6-(6-chloro-4-(cyclopentylamino)-1H-pyrazolo[3,4-d]pyrimidin-1-yl)-2,2-dimethyltetrahydrofuro[3,4-d][1,3]dioxol-4-yl)methoxy)-3-hydroxypropan-2-yl)phosphonate (1k) (189 mg, 0.269 mmol) in MeCN (13.5 mL) was added triethylamine (0.751 mL, 5.38 mmol) followed by bromotrimethylsilane (0.528 mL, 4.04 mmol) at rt under argon atmosphere. After the solution was stirred at rt for 4 h, it was concentrated under reduced pressure. The residue was dissolved in TFA/water (1/3, 10 mL) and it was stirred at rt for 2 h. The mixture was concentrated under reduced pressure and the residue was purified by reverse phase HPLC (15-40% ACN/H₂O, 0.1% TFA, a gradient elution) to provide the title compound (1) as an off-white solid (TFA salt, 107 mg, 55%). ¹H NMR (400 MHz, methanol-d₄) δ 8.08 (d, J=0.8 Hz, 1H), 6.25-6.20 (m, 1H), 4.96 (s, 2H), 4.72-4.69 (m, 1H), 4.57-4.47 (m, 211), 4.19-4.16 (m, 111), 4.08 (dd, J=10.4, 4.0 Hz, 111), 4.01-3.92 (m, 4H), 3.84 (dd, J=12.4, 7.6 Hz, 1H), 2.13-2.06 (m, 2H), 1.84-1.57 (m, 6H); m/z (ESI, +ve ion)=606.1 [M+H]⁺.

Alternatively, Example 1, ((S)-1-((2H-tetrazol-5-yl)methoxy)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-hydroxypropan-2-yl)phosphonic acid, was prepared by Steps M to O below.

### Step M. tert-butyl (6-chloro-1-((3aR,4R,6R,6aR)-6-((((R)-2-(diethoxyphosphoryl)-1-hydroxy-3-(2-(trimethylsilyl)ethoxy)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (1m)

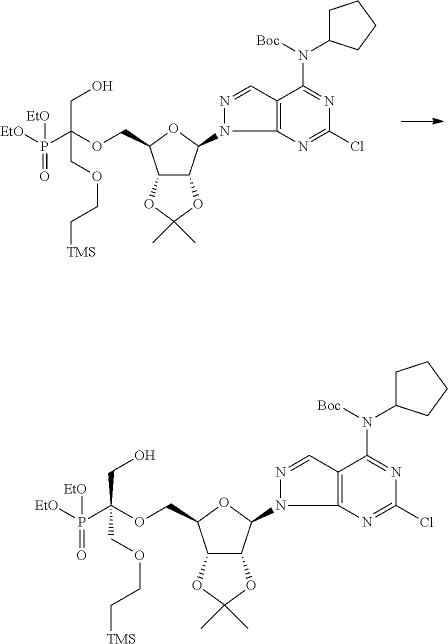

Diastereomers from Step H (1h) were separated by chiral chromatography (CHIRALPAK, AD-H, 21×250 mm, 5 μm, 5% IPA/hexanes, an isocratic elution, a flow rate of 20 mL/min, and the second eluted isomer was identified as the title compound (1m) and was collected.

### Step N. tert-butyl (6-chloro-1-((3aR,4R,6R,6aR)-6-((((R)-2-(diethoxyphosphoryl)-1-(2-(trimethylsilyl)ethoxy)-3-((2-((2-(trimethylsilyl)ethoxy)methyl)-2H-tetrazol-5-yl)methoxy)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (In)

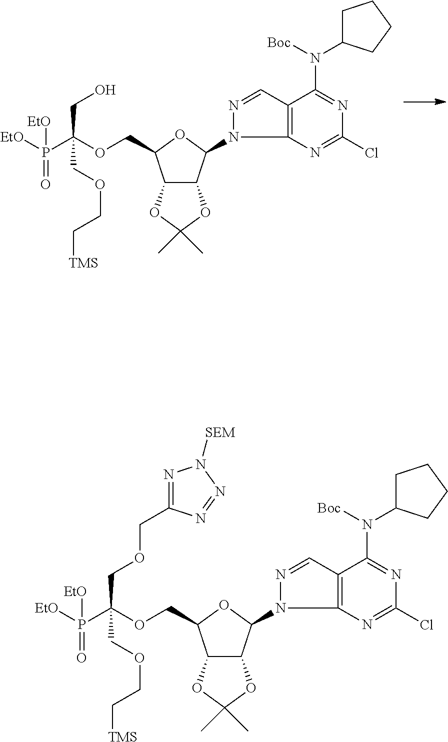

To a solution of tert-butyl (6-chloro-1-((3aR,4R,6R,6aR)-6-((((R)-2-(diethoxyphosphoryl)-1-hydroxy-3-(2-(trimethylsilyl)ethoxy)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (1m) (2.07 g, 2.52 mmol) and 5-(bromomethyl)-2-((2-(trimethylsilyl)ethoxy)methyl)-2H-tetrazole (2.96 g, 10.1 mmol) in DMF (12.5 mL) at 0 °C was added NaH (60% mineral oil, 252 mg, 6.31 mmol) in one portion. After the mixture was stirred at 0 °C for 30 min, the mixture was quenched by sat. aq. NH₄Cl, diluted with EtOAc and water. The solution was extracted (EtOAc) and the combined organic layers were washed (brine), dried (Na₂SO₄), and concentrated under reduced pressure. The resulting residue was purified by flash chromatography (5-30% acetone/hexanes, a gradient elution) to afford the title compound (In) (2.2 g, 84%) as a light-yellow gum.

### Step O. ((S)-1-((2H-tetrazol-5-yl)methoxy)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-hydroxypropan-2-yl)phosphonic acid (1)

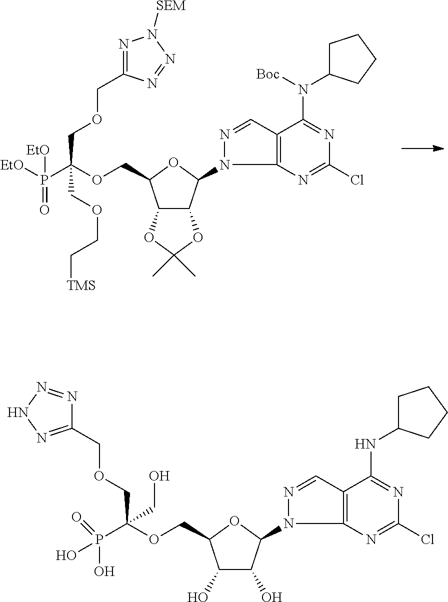

To a solution of tert-butyl (6-chloro-1-((3aR,4R,6R,6aR)-6-((((R)-2-(diethoxyphosphoryl)-1-(2-(trimethylsilyl)ethoxy)-3-((2-((2-(trimethylsilyl)ethoxy)methyl)-2H-tetrazol-5-yl)methoxy)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (In) (8.00 g, 7.75 mmol) in MeCN (300 mL) was added triethylamine (16.2 mL, 116 mmol) followed by bromotrimethylsilane (10.1 mL, 77.5 mmol) at rt under argon atmosphere. After the solution was stirred for 15 h, it was concentrated under reduced pressure and azeotropically distilled with toluene (2 times). The residue was partitioned between EtOAc and water. The organic layer was collected, and the aqueous layer was extracted two times with EtOAc. The combined organic layers were washed once more with water and concentrated under reduced pressure. The crude solid was dissolved in TFA/water (1/1, 280 mL) and it was stirred at rt for 24 h. The mixture was concentrated under reduced pressure and the residue was purified by reverse phase HPLC (15-35% ACN/H₂O, 0.1% TFA, a gradient elution) to provide the title compound (1) as a white solid (TFA salt, 3.1 g, 56%). ¹H NMR (400 MHz, methanol-d₄) δ 8.08 (d, J=0.8 Hz, 1H), 6.25-6.20 (m, 1H), 4.96 (s, 2H), 4.72-4.69 (m, 1H), 4.57-4.47 (m, 2H), 4.19-4.16 (m, 1H), 4.08 (dd, J=10.4, 4.0 Hz, 1H), 4.01-3.92 (m, 4H), 3.84 (dd, J=12.4, 7.6 Hz, 1H), 2.13-2.06 (m, 2H), 1.84-1.57 (m, 6H); m/z (ESI, +ve ion)=606.1 [M+H]⁺.

### Example 2: Preparation of Succinate Form of Compound 1

A slurry was prepared comprising an amount of Compound 1 (20 mg, free base) and an equimolar amount of succinic acid in tetrahydrofuran/water (about 0.2 mL, 9:1, v/v) and the resulting slurry was stirred at room temperature for 3 days and then at 5 °C for an additional 3 days resulting in a clear solution. An amount of acetonitrile (about 5 mL) was then added, resulting in a clear solution which was transferred to an open vessel. The solvents were allowed to evaporate to provide a solid precipitate that was collected to afford a succinate form of Compound 1.

### Example 3: Preparation of Form 1 of Gentisate Form of Compound 1

Into a vial were placed an amount of Compound 1 (20 mg, free base) and an equimolar amount of gentisic acid in THF/H2O (9:1, v/v) and the resulting mixture was stirred for three days at room temperature and then for another three days at 5 °C for 3 days, resulting in a clear solution. An amount of acetonitrile (about 5 mL) was then added, resulting in a clear solution which was transferred to an open vessel. The solvents were allowed to evaporate to provide a solid precipitate that was collected to afford Form 1 of a gentisate form of Compound 1.

### Example 4: Preparation of Form 2 of Gentisate Form of Compound 1

Into a vial were placed an amount of Compound 1 (50.2 mg, free base) and gentisic acid (12.5 mg) were added into a 5-mL glass vial, to which were added 1 mL of a mixture of tetrahydrofuran/water (9:1, v/v) into the vial to afford a slurry that was stirred at room temperature for 1 day until a clear solution was obtained. An amount of acetonitrile (3 mL) was added to the solution to afford a clear solution. The solvents were then allowed to evaporate at room temperature to provide a solid precipitate that was collected to afford Form 2 of a gentisate form of Compound 1.

### Example 5: Preparation of Form 2 of Gentisate Form of Compound 1

To a solution of the trifluoracetic acid form of Compound 1 from Example 1 in a mixture of water/n-propanol (2:98, 3 volumes) was added gentisic acid (3 molar equivalents) and the resulting mixture was stirred at 25 °C. To the resulting solution was added a seed of Form 2 gentisate form of Compound 1 (which may be prepared according to methods similar to Example 4) (1 mol %) and the resulting mixture was stirred for >1 h. Heptane (2 volumes) was then added over >1 h and the resulting mixture was stirred for >12 h. Additional heptane (19 volumes) was added to the mixture over >10 h, and the mixture was stirred for >8 h. The mixture was filtered, the solid washed three times with a water/n-propanol/heptane mixture (1:37:100, 2 volumes), deliquored, and dried at 40 °C under vacuum and with the humidity inside the dryer controlled to 25% to 45% relative humidity to afford Form 2 of a gentisate form of Compound 1.

### Example 6: X-Ray Powder Diffraction (XRPD) Analysis of Polymorphic Forms of a Gentisate Form of Compound 1

XRPD analyses of polymorphic forms of gentisate forms of Compound 1 were performed using Panalytical Empyrean and X'pert3 X-ray powder diffractometers. Samples were spread on the middle of a zero-background Si holder. The parameters used for the analyses are set forth in Table 2.

TABLE 2

| Parameter | Reflection Mode | VT-XRPD |
|---|---|---|
| Model | X′Pert³ | Empyrean |
| X-ray wavelength | Cu, kα | |
| | Kα1 (angstroms): 1.540598; | |
| | Kα2 (angstroms): 1.544426 | |
| | Kα2/Kα1 intensity ratio: 0.50 | |
| X-ray tube setting | 45 kV, 40 mA | 45 kV, 40 mA |
| Divergence slit | 1/8° | 1/8° |
| Scan mode | Continuous | Continuous |
| Scan range (°2TH) | 3° to 40° | 3° to 40° |
| Scan step time (s) | 46.67 | 33.02 |
| Step size (°2TH) | 0.0263 | 0.0167 |
| Test time | About 5 minutes, | About 10 minutes, |
| | 4 seconds | 15 seconds |

Form 1 of a gentisate form of Compound 1 was analyzed by XRPD as set forth above and exhibited the peaks set forth in Table 3. The error associated with each °2theta position was determined to be ±0.2°theta.

TABLE 3

| Position | | FWHM Left | d-spacing | Relative |
|---|---|---|---|---|
| (°2theta) | Height | (°2theta) | (angstroms) | intensity (%) |
| 8.26 | 1846.80 | 0.1279 | 10.71 | 100.0 |
| 13.25 | 132.81 | 0.1535 | 6.68 | 7.19 |
| 14.17 | 157.35 | 0.1535 | 6.25 | 8.52 |
| 14.94 | 259.58 | 0.2047 | 5.93 | 14.06 |
| 15.40 | 336.54 | 0.1279 | 5.75 | 18.22 |
| 15.81 | 392.44 | 0.1279 | 5.60 | 21.25 |
| 16.36 | 182.92 | 0.2047 | 5.42 | 9.90 |
| 18.15 | 174.00 | 0.2047 | 4.89 | 9.42 |
| 19.61 | 42.33 | 0.3070 | 4.53 | 2.29 |
| 20.44 | 260.98 | 0.1023 | 4.34 | 14.13 |
| 21.56 | 55.83 | 0.3070 | 4.12 | 3.02 |
| 26.43 | 488.28 | 0.2047 | 3.37 | 26.44 |
| 27.59 | 255.28 | 0.1535 | 3.23 | 13.82 |
| 29.81 | 49.88 | 0.6140 | 3.00 | 2.70 |
| 31.22 | 74.01 | 0.3070 | 2.86 | 4.01 |

Form 2 of a gentisate form of Compound 1 was analyzed by XRPD as set forth above and exhibited the peaks set forth in Table 4. The error associated with each °2theta position was determined to be ±0.2°theta.

TABLE 4

| Position | | FWHM Left | d-spacing | Relative |
|---|---|---|---|---|
| (°2theta) | Height | (°2theta) | (angstroms) | intensity (%) |
| 4.64 | 67.51 | 0.3070 | 19.04 | 13.12 |
| 6.97 | 240.68 | 0.1279 | 12.68 | 46.78 |
| 9.25 | 514.45 | 0.2047 | 9.56 | 100.00 |
| 10.36 | 101.29 | 0.1535 | 8.54 | 19.69 |
| 13.32 | 99.70 | 0.2558 | 6.65 | 19.38 |
| 14.61 | 156.13 | 0.1535 | 6.60 | 30.35 |
| 18.89 | 203.35 | 0.2047 | 4.70 | 39.53 |
| 20.53 | 333.31 | 0.1791 | 4.33 | 64.79 |
| 22.60 | 100.94 | 0.2047 | 3.93 | 19.62 |
| 23.62 | 69.72 | 0.2047 | 3.77 | 13.55 |
| 26.08 | 310.62 | 0.4605 | 3.42 | 60.38 |
| 28.30 | 108.54 | 0.8187 | 3.15 | 21.10 |
| 34.62 | 34.16 | 0.4093 | 2.59 | 6.64 |

### Example 7: Thermal Gravimetric Analyses and Differential Scanning Calorimetry Analyses of a Gentisate Form of Compound 1

Thermal gravimetric analysis (TGA) data were collected using a TA Q5000 and Discovery TGA 5500 TGA from TA Instruments. Differential scanning calorimetry (DSC) analyses were performed using a TA Q2000 DSC from TA Instruments using the parameters set forth in Table 5.

TABLE 5

| Parameter | TGA | DSC |
|---|---|---|
| Method | Ramp | Ramp |
| Sample pan | Aluminum, open | Aluminum, open |
| Temperature | Room temperature | 25 °C to |
| range | to about 350 °C | about 200 °C |
| Heating rate | 10 °C/minute | 10 °C/minute |
| Purge gas | Nitrogen | Nitrogen |

### Example 8: Solubility of Polymorphic Forms of a Gentisate Form of Compound 1

The solubility of a polymorphic form of a gentisate form of Compound 1 was measured in water, simulated gastric fluid (SGF), fasted-state simulated intestinal fluid (FaSSIF), and fed-state simulated intestinal fluid (FeSSIF) after 4 hours and at 37 °C as follows.

The SGF media was prepared by weighing 49.5 mg of NaCl and 25.4 mg of Triton X-100 into a 100-mL volumetric flask. A volume of purified water was added to the flask and the resulting mixture was sonicated until all solids were dissolved. About 1.632 mL of HCl solution (1 M) were then added and sufficient purified water to the target volume and to adjust the pH to 1.8. The solution was then diluted to volume with purified water, mixed well and the pH value was measured to be 1.83.

A FaSSIF buffer was prepared by weighing 340.8 mg of NaH₂PO₄, 43.0 mg of NaOH and 619.6 mg of NaCl into a 100-mL volumetric flask. A volume of purified water was added to the flask and the resulting mixture was sonicated until the solids had dissolved. A second volume of purified water was added to the flask to adjust the pH to 6.5. The solution was diluted with another volume of purified water, mixed, and the pH value was measured to be 6.54. The FaSSIF media was prepared by weighing 110.4 mg of SIF powder into a 50-mL volumetric flask to which a volume of FaSSIF dissolving buffer was added. The resulting mixture was sonicated until the SIF powder dissolved. Then mixture was then diluted to volume with FaSSIF dissolving buffer and mixed well. The FaSSIF solution was equilibrated for 2 hours at room temperature before it was used.

A FeSSIF dissolving buffer was prepared by weighing 0.82 mL of glacial acetic acid, 404.9 mg of NaOH and 1188.2 mg of NaCl into a 100-mL volumetric flask. A volume of purified water was added to the flask and the mixture was sonicated until the solids were dissolved. A second volume of purified water was added to the target volume to adjust the pH to 5.0. The solution was diluted with a volume of purified water, mixed well the pH value was measured to be 4.96. FeSSIF media was prepared by weighing 559.6 mg of SIF powder into a 50-mL volumetric flask. A volume of FeSSIF dissolving buffer was added to the flask and the resulting mixture was sonicated the SIF powder was dissolved. A second volume FeSSIF dissolving buffer was added and the resulting mixture was mixed well. The FeSSIF solution was equilibrated for 2 hours to room temperature before use.

An Agilent 1260 high performance liquid chromatography (HPLC) instrument equipped with a DAD detector, a Waters H-Class UPLC with PDA detector was used in the solubility measurements using the conditions set forth in Table 6.

TABLE 6

| | |
|---|---|
| Parameter | Value |

| Column | Supelco Ascentis Express | |
|---|---|---|
| | 2.7 μm 4.6 mm × 100 mm | |
| Mobile phase | A: 0.1% trifluoroacetic acid in water | |
| | B: 0.1% trifluoroacetic acid in acetonitrile | |
| Solvent gradient | Run time 0 minutes: 2% B | |
| | Run time 5 minutes: 30% B | |
| | Run time 7 minutes: 90% B | |
| | Run time 7.01 minutes: 2% B | |
| | Run time 8 minutes: 2% B | |
| Run time | 8.0 | minutes |
| Post time | 0.0 | |
| Flow rate | 0.6 | mL/minute |
| Injection volume | 10 | μL |
| Detector wavelength | 220 nm, 280 nm | |
| Column temperature | 35 | °C |

| | |
|---|---|
| Sampler temperature | Room temperature |
| Diluent | Methanol |

About 10 mg of a gentisate Form 2 of Compound 1 (calculated by weight of the free base of Compound 1) was placed into a 3 mL glass vial and 1 mL of the respective media (water, SGF, FaSSIF and FeSSIF) was added into each glass vial. A cap was placed on each of the vials and they were rolled at 37 °C (25 rpm) for 4 hours. The suspension was then extracted into a centrifugation tube prior to centrifugation (10000 rpm, 37 °C, 5 min) and filtration (0.22 μm PTFE membrane). The resulting supernatant was analyzed by HPLC and the pH was determined. The solubility of the polymorphic form of a gentisate Form 2 of Compound 1 measured was as set forth above was as set forth in Table 7.

TABLE 7

| | Solubility (mg/mL) | |
|---|---|---|
| | (calculated as the | |
| | amount of free base | |
| Media | of Compound 1) | pH |
| Water | 26.20 | 1.7 |
| Simulated gastric | >9.71 | 1.7 |
| fluid (SGF) | | |
| fasted-state simulated | >10.24 | 2.5 |
| intestinal fluid (FaSSIF) | | |
| fed-state simulated | >9.42 | 4.4 |
| intestinal fluid (FeSSIF) | | |

### Example 9: Tablets Comprising a Gentisate Form of Compound 1

Tablets comprising 261 mg of a Form 2 of a gentisate form of Compound 1 (200 mg of the free base of Compound 1) were prepared according to the following method. The quantities of each component used were as set forth in Table 8, each of which other than magnesium stearate and sodium stearyl fumarate were passed through a 420 mesh sieve before use. Magnesium stearate and sodium stearyl fumarate were passed through a #35 mesh sieve before use.

TABLE 8

| | | Target weight | Dispensed |
|---|---|---|---|
| | Target amount | percent of | sieved |
| Material | per tablet (mg) | each tablet | quantity (grams) |
| Compound 1 | 261.438 | 38.45% | 784.32 |
| gentisate form | | | |
| Microcrystalline | 377.162 | 55.46% | 1131.5 |
| cellulose PH-102 | | | |
| First colloidal | 3.500 | 0.515% | 10.52 |
| silica dioxide | | | |
| Aerosil 200 Pharma | | | |
| First sodium | 3.500 | 0.515% | 10.51 |
| stearyl fumarate | | | |
| First crospovidone | 10.200 | 1.50% | 30.61 |
| Kollidon CL | | | |
| First Magnesium | 3.500 | 0.515% | 10.51 |
| stearate | | | |
| Second sodium | 3.500 | 0.515% | 10.51 |
| stearyl fumarate | | | |
| Second colloidal | 3.500 | 0.515% | 10.50 |
| silica dioxide | | | |
| Aerosil 200 Pharma | | | |
| Second cropovidone | 10.200 | 1.50% | 30.60 |
| Kollidon CL | | | |
| Second magnesium | 3.500 | 0.515% | 10.51 |
| stearate | | | |
| Total | 680 | 100% | 2040.09 |

Into a 10 liter bin were placed one-third of sieved microcrystalline cellulose which was blended at a 20 rpm for 5 minutes. To the bin were then added an additional one-third of the microcrystalline cellulose, a gentisate form of Compound 1, and the remaining one-third of the microcrystalline cellulose (which was used to rinse the bag that contained the gentisate form of Compound 1), the first portion of crospovidone Kollidon CL, the first portion of colloidal silicon dioxide Aerosil 200 Pharma, and the first portion of sodium stearyl fumarate. The resulting mixture was then blended at 20 rpm for 20 minutes. The blended mixture was passed through a 420 mesh sieve and the sieved mixture was added to a 10 liter bin and further blended at 20 rpm for 15 minutes. The first portion of the magnesium stearate was then added to the center of the blended mixture and further blended at 20 rpm for 5 minutes. The resulting mixture was then discharged into low-density polyethylene bags.

A roller compactor was equipped with the following settings: (a) roller width: 40 mm; (b) upper roller surface: knurled; (c) lower roller surface: knurled; (d) coarse RFG screen: 2.0 mm, wired; (e) fine RFG screen: 1.0 mm, wired. The feed hopper of the roller compactor was then charged with the blended mixture from above and the materials were processed using the parameters for the roller compactor set forth in Table 9. The granules (1454.25 g) were collected into low-density polyethylene bags.

TABLE 9

| Parameter | Setting |
|---|---|
| Roller pressure (MPa) | 1.8 |
| Rolling gap (mm) | 3 |
| Roller speed (rpm) | 4 |
| Granulator (RFG) speed (rpm) | Fixed |
| Feed screw speed (rpm) | 30 |
| Gap control | Off |
| Un-compacted fines | Recycle |
| Envelope density | 1.0046 g/mL |
| Solid fraction | 0.653 |

The granules produced by the roller compactor (1454.25 g) were placed into a 10 liter bin, to which were added sodium stearyl fumarate (7.725 g), colloidal silica dioxide Aerosil 200 Pharma (7.78 g), crospovidone Kollidon CL (22.503 g), and the resulting mixture was blended at 20 rpm for 15 minutes. To the blended mixture was added magnesium stearate (7.72 g) and the mixture was blended at 20 rpm for 5 minutes to afford a final blend.

A tableting machine was equipped with the following: (a) upper punch, lower punch, and die=19 mm by 8.51 mm; (b) punch numbers=2; and (c) fill cam size=8 mm to 14 mm. The tableting machine was set with the parameters set forth in Table 10 to afford tablets that were within the target parameters set forth in Table 11.

TABLE 10

| Compression parameter | Setting |
|---|---|
| Rotary speed (rpm) | 20 |
| Feed speed (rpm) | 27 |
| Main compression (kN) | 11.7 |
| Fill depth (mm) | 8.7 |

TABLE 11

| Target parameter | Target value |
|---|---|
| Individual tablet weight (mg) | 663 to 714 |
| Tablet thickness | 5.4 mm to 5.5 mm |
| Tablet hardness | 170 N to 220 N |
| Tablet friability | Not more than 1% |
| Disintegration time | Less than 15 minutes |

### Example 10: Preparation of Form 2 of a Gentisate Form of Compound 1

To a reactor under nitrogen at about 25 °C were placed 2.8 kg of a 2:98 mixture of water/n-propanol and 1.73 kg of the trifluoroacetic acid form of Compound 1. The resulting mixture was stirred at a temperature of about 25 °C until the solids dissolved, after which 1.31 kg of 2,5-dihydroxybenzoic acid (1.31 kg) was added, followed by additional portions of the 2:98 water/n-propanol mixture. The resulting mixture was stirred at about 25 °C until the solids dissolved, after which a portion of Form 2 of a gentisate form of Compound 1 (about 0.02 kg) was added to the mixture, which was stirred at about 25 °C for an additional period of about 30 minutes. To the resulting mixture was added n-heptane (about 22 kg) and the resulting mixture was stirred at about 25 °C for an additional period of about 16 hours. The resulting solids were filtered, washed, and dried under vacuum in an oven set to a temperature of about 40 °C to about 50 °C and at relative humidity of about 30% to about 40% to provide Form 2 of a gentisate form of Compound 1 (about 2 kg).

### Example 11: X-Ray Powder Diffraction (XRPD) Analysis of Form 2 of a Gentisate Form of Compound 1

A sample of Form 2 of a gentisate form of Compound 1 that was prepared using a method analogous to that described in Example 10 was analyzed by XRPD and exhibited the peaks set forth in Table 12. The error associated with each °2theta position was determined to be ±0.2°theta.

TABLE 12

| Position | d-spacing | Relative |
|---|---|---|
| (°2theta) | (angstroms) | intensity (%) |
| 4.8 | 18.5 | 16.8 |
| 7.1 | 12.5 | 43.9 |
| 7.6 | 11.6 | 7.5 |
| 9.3 | 9.5 | 100 |
| 10.5 | 8.4 | 19.1 |
| 13.4 | 6.6 | 12.8 |
| 13.8 | 6.4 | 10.4 |
| 14.4 | 6.1 | 15.1 |
| 14.8 | 6.0 | 19.1 |
| 17.2 | 5.1 | 6.5 |
| 17.7 | 5.0 | 7.7 |
| 18.8 | 4.7 | 12.3 |
| 19.0 | 4.7 | 26.3 |
| 20.1 | 4.3 | 35.5 |
| 21.2 | 4.2 | 11.5 |
| 21.8 | 4.1 | 7.8 |
| 22.7 | 3.9 | 12.4 |
| 23.7 | 3.7 | 8.9 |
| 26.2 | 3.4 | 32.2 |
| 27.9 | 3.2 | 10.1 |
| 28.3 | 3.2 | 12.1 |
| 34.7 | 2.6 | 4.0 |
| 35.6 | 2.5 | 3.2 |
| 35.7 | 2.5 | 3.7 |

### Example 12: Stability of Form 2 of a Gentisate Form of Compound 1

To measure stability of the Form 2 of a gentisate form of Compound 1 under storage conditions, samples of Form 2 of a gentisate form of Compound 1 were placed in double low-density polyethylene bags with desiccant between the bags and each was placed into a high-density polyethylene drum. One drum was stored at a temperature of 5 °C and samples of the material were taken at 1 month and 3 months, and the samples were analyzed for the presence of impurities. The other drum was stored at a temperature of 25 °C and at 60% relative humidity (RH), samples of the materials were taken at 1 month and 3 months, and the samples were analyzed for the presence of impurities. The amount of the Form 2 of a gentisate form of Compound 1, and the amount ofany impurities, in each sample was measured by reverse-phase high performance liquid chromatography using the test conditions set and solvent gradients forth in Table 13 and Table 14. The samples were tested to determine the amount remaining of Form 2 at each time point, and the measurements were conducted by x-ray powder diffraction (XRPD) according to USP <941>.

TABLE 13

| Column | YMC-Triart PFP, 4.6 × 150 mm × 3.0 μm | |
|---|---|---|
| Flow (mL/min) | 1.5 | mL/min |
| Run time (min) | 50 | min |
| Injector | 5 | μL |
| volume (μL) | | |
| Column | 35 | °C |
| temperature (°C) | | |
| Detection wavelength | 220 | nm |

| | |
|---|---|
| Compound 1 | 18.80 |
| retention time (min) | |
| Needle wash | Acetonitrile |
| Mobile Phase A | 0.1% H₃PO₄ in water (v/v) |
| Mobile Phase B | 0.1% H₃PO₄ in methanol:acetonitrile (1:1, v/v) |
| Diluent | Acetonitrile:water (50:50, v/v) |

TABLE 14

| Time | Mobile | Mobile |
|---|---|---|
| (min) | Phase A % | Phase B % |
| Initial | 90 | 10 |
| 10 | 80 | 20 |
| 13 | 80 | 20 |
| 30 | 65 | 35 |
| 40 | 0 | 100 |
| 45 | 0 | 100 |
| 45.10 | 90 | 10 |
| 50 | 90 | 10 |

The results of the stability tests for Form 2 of a gentisate form of Compound 1 under both storage conditions is set forth in Table 15.

TABLE 15

| | | | |
|---|---|---|---|
| | Initial | Time = | Time = |
| Test Parameter | (Time = zero) | 1 month | 3 months |

Storage Conditions = 5 °C

| | | | |
|---|---|---|---|
| Total impurities present | 4.4 | 4.5 | 4.2 |
| (% area measured by | | | |
| reverse-phase HPLC) | | | |
| XRPD | Crystalline | Crystalline | Crystalline |

Storage Conditions = 25 °C and at 60% RH

| | | | |
|---|---|---|---|
| Total impurities present | 4.4 | 4.5 | 4.3 |
| (% area measured by | | | |
| reverse-phase HPLC) | | | |
| XRPD | Crystalline | Crystalline | Crystalline |

The results demonstrated that Form 2 of a gentisate form of Compound 1 was stable up to 3 months when stored at 5 °C, and was stable up to 3 months when stored at 25 °C and at 60% RH.

### EMBODIMENTS

### Embodiment 1: A Pharmaceutically Acceptable Form of Compound 1

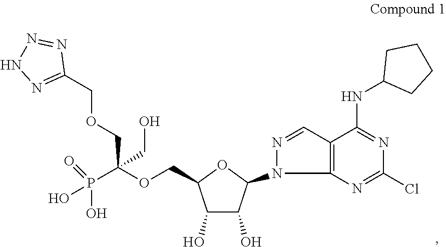 [SMILES: O=P(O)(O)[C@@](CO)(COCc1nn[nH]n1)OC[C@H]1O[C@@H](n2ncc3c(NC4CCCC4)nc(Cl)nc32)[C@H](O)[C@@H]1O]

wherein the pharmaceutically acceptable from is selected from a gentisate form and a succinate form.

Embodiment 2: The pharmaceutically acceptable form of embodiment 1, wherein the pharmaceutically acceptable form is a gentisate form.

Embodiment 3: The pharmaceutically acceptable form of embodiment 2, wherein the gentisate form comprises a gentisate salt.

Embodiment 4: The pharmaceutically acceptable form of embodiment 2, wherein the gentisate form comprises a gentisate co-crystal.

Embodiment 5: The pharmaceutically acceptable form of any one of embodiments 1 to 4, wherein in the gentisate form the molar ratio between Compound 1 and gentisic acid is about 1:1.

Embodiment 6: The pharmaceutically acceptable form of any one of embodiments 1 to 5, wherein the gentisate form is a hydrate.

Embodiment 7: The pharmaceutically acceptable form of embodiment 6, wherein the hydrate is selected from a hemi-hydrate, a mono-hydrate, and a di-hydrate.

Embodiment 8: The pharmaceutically acceptable form of embodiment 7, wherein the hydrate is a hemi-hydrate.

Embodiment 9: The pharmaceutically acceptable form of embodiment 7, wherein the hydrate is a mono-hydrate.

Embodiment 10: The pharmaceutically acceptable form of embodiment 7, wherein the hydrate is a di-hydrate.

Embodiment 11: The pharmaceutically acceptable form of any one of embodiments 1 to 10, wherein the pharmaceutically acceptable form is a solid.

Embodiment 12: The pharmaceutically acceptable form of embodiment 11, wherein the solid is a crystalline solid.

Embodiment 13: The pharmaceutically acceptable form of embodiment 12, wherein the crystalline solid exhibits a peak in an x-ray powder diffraction (XRPD) pattern at 9.25±0.2° 2-theta.

Embodiment 14: The pharmaceutically acceptable form of embodiment 13, wherein the crystalline solid exhibits further peaks in an x-ray powder diffraction (XRPD) pattern at 6.97±0.2° 2-theta, 20.53±0.2° 2-theta, and 26.08±0.2° 2-theta.

Embodiment 15: The pharmaceutically acceptable form of embodiment 13 or 14, wherein the crystalline solid exhibits further peaks in an x-ray powder diffraction (XRPD) pattern at 14.61±0.2° 2-theta and 18.89±0.2° 2-theta.

Embodiment 16: The pharmaceutically acceptable form of any one of embodiments 12 to 15, wherein the crystalline solid further comprises a peak in a differential scanning calorimetry pattern of from about 150 °C to about 170 °C.

Embodiment 17: The pharmaceutically acceptable form of embodiment 16, wherein the crystalline solid comprises a peak in a differential scanning calorimetry pattern of from about 150 °C to about 165 °C.

Embodiment 18: The pharmaceutically acceptable form of embodiment 16, wherein the crystalline solid comprises a peak in a differential scanning calorimetry pattern from about 161 °C to about 162 °C.

Embodiment 19: The pharmaceutically acceptable form of any one of embodiments 12 to 18, the crystalline solid exhibits a loss in mass in a thermal gravimetric analysis of between about 1% to about 5% upon heating from about 31 °C to about 150 °C.

Embodiment 20: The pharmaceutically acceptable form of embodiment 19, wherein the crystalline solid further exhibits a loss in mass in a thermal gravimetric analysis of between about 3% to about 5% upon heating from about 31 °C to about 150 °C.

Embodiment 21: The pharmaceutically acceptable form of embodiment 19, wherein the crystalline solid further exhibits a loss in mass in a thermal gravimetric analysis of about 5% upon heating from about 31 °C to about 150 °C.

Embodiment 22: The pharmaceutically acceptable form of any one of embodiments 1 to 21, wherein the pharmaceutically acceptable form exhibits a solubility of at least 5 mg/mL in an aqueous solution having a pH of 1.7 and a temperature of 37 °C.

Embodiment 23: The pharmaceutically acceptable form of embodiment 22, wherein the pharmaceutically acceptable form exhibits a solubility of at least 10 mg/mL in an aqueous solution having a pH of 1.7 and a temperature of 37 °C.

Embodiment 24: The pharmaceutically acceptable form of embodiment 23, wherein the pharmaceutically acceptable form exhibits a solubility of at least 15 mg/mL in an aqueous solution having a pH of 1.7 and a temperature of 37 °C.

Embodiment 25: The pharmaceutically acceptable form of embodiment 23, wherein the pharmaceutically acceptable form exhibits a solubility of at least 20 mg/mL in an aqueous solution having a pH of 1.7 and a temperature of 37 °C.

Embodiment 26: The pharmaceutically acceptable form of embodiment 23, wherein the pharmaceutically acceptable form exhibits a solubility of at least 25 mg/mL in an aqueous solution having a pH of 1.7 and a temperature of 37 °C.

Embodiment 27: The pharmaceutically acceptable form of any one of embodiments 1 to 21, wherein the pharmaceutically acceptable form exhibits a solubility of between about 10 mg/mL and about 30 mg/mL in an aqueous solution having a pH of 1.7 and a temperature of 37 °C.

Embodiment 28: The pharmaceutically acceptable form of embodiment 27, wherein the pharmaceutically acceptable form exhibits a solubility of between about 15 mg/mL and about 30 mg/mL in an aqueous solution having a pH of 1.7 and a temperature of 37 °C.

Embodiment 29: The pharmaceutically acceptable form of embodiment 27, wherein the pharmaceutically acceptable form exhibits a solubility of between about 20 mg/mL and about 30 mg/mL in an aqueous solution having a pH of 1.7 and a temperature of 37 °C.

Embodiment 30: The pharmaceutically acceptable form of embodiment 27, wherein the pharmaceutically acceptable form exhibits a solubility of between about 25 mg/mL and about 30 mg/mL in an aqueous solution having a pH of 1.7 and a temperature of 37 °C.

Embodiment 31: The pharmaceutically acceptable form of any one of embodiments 1 to 30, wherein the pharmaceutically acceptable form exhibits a solubility of at least 5 mg/mL in an aqueous solution having a pH of 2.5 and a temperature of 37 °C.

Embodiment 32: The pharmaceutically acceptable form of embodiment 31, wherein the pharmaceutically acceptable form exhibits a solubility of at least 7.5 mg/mL in an aqueous solution having a pH of 2.5 and a temperature of 37 °C.

Embodiment 33: The pharmaceutically acceptable form of embodiment 31, wherein the pharmaceutically acceptable form exhibits a solubility of at least 10 mg/mL in an aqueous solution having a pH of 2.5 and a temperature of 37 °C.

Embodiment 34: The pharmaceutically acceptable form of any one of embodiments 1 to 33, wherein the pharmaceutically acceptable form exhibits a solubility of at least 1 mg/mL in an aqueous solution having a pH of 4.4 and a temperature of 37 °C.

Embodiment 35: The pharmaceutically acceptable form of embodiment 34, wherein the pharmaceutically acceptable form exhibits a solubility of at least 2.5 mg/mL in an aqueous solution having a pH of 4.4 and a temperature of 37 °C.

Embodiment 36: The pharmaceutically acceptable form of embodiment 34, wherein the pharmaceutically acceptable form exhibits a solubility of at least 5 mg/mL in an aqueous solution having a pH of 4.4 and a temperature of 37 °C.

Embodiment 37: The pharmaceutically acceptable form of embodiment 34, wherein the pharmaceutically acceptable form exhibits a solubility of at least 7.5 mg/mL in an aqueous solution having a pH of 4.4 and a temperature of 37 °C.

Embodiment 38: The pharmaceutically acceptable form of any one of embodiments 1 to 37, wherein the pharmaceutically acceptable form exhibits less than about 10% degradation when the pharmaceutically acceptable form is stored at 25 °C and 60% relative humidity for at least 7 days.

Embodiment 39: The pharmaceutically acceptable form of embodiment 38, wherein the pharmaceutically acceptable form exhibits less than about 1% degradation when the pharmaceutically acceptable form is stored at 25 °C and 60% relative humidity for at least 7 days.

Embodiment 40: The pharmaceutically acceptable form of any one of embodiments 1 to 39, wherein the pharmaceutically acceptable form exhibits less than about 10% degradation when the pharmaceutically acceptable form is stored at 40 °C and 75% relative humidity for at least 7 days.

Embodiment 41: The pharmaceutically acceptable form of embodiment 40, wherein the pharmaceutically acceptable form exhibits less than about 1% degradation when the pharmaceutically acceptable form is stored at 40 °C and 75% relative humidity for at least 7 days.

Embodiment 42: The pharmaceutically acceptable form of any one of embodiments 1 to 41, wherein the pharmaceutically acceptable form exhibits less than about 10% degradation when the pharmaceutically acceptable form is stored at 60 °C for one or more day.

Embodiment 43: The pharmaceutically acceptable form of embodiment 42, wherein the pharmaceutically acceptable form exhibits less than about 1% degradation when the pharmaceutically acceptable form is stored at 60 °C for one or more day.

Embodiment 44: The pharmaceutically acceptable form of embodiment 12, wherein the crystalline solid exhibits a peak in an x-ray powder diffraction (XRPD) pattern at 8.26±0.2° 2-theta.

Embodiment 45: The pharmaceutically acceptable form of embodiment 44, wherein the crystalline solid exhibits a further peak in an x-ray powder diffraction (XRPD) pattern at 26.43±0.2° 2-theta.

Embodiment 46: The pharmaceutically acceptable form of embodiment 44 or 45, wherein the crystalline solid exhibits further peaks in an x-ray powder diffraction (XRPD) pattern at 15.81±0.2° 2-theta and 15.40±0.2° 2-theta.

Embodiment 47: The pharmaceutically acceptable form of any one of embodiments 44 to 46, wherein the crystalline solid exhibits further peaks in an x-ray powder diffraction (XRPD) pattern at 14.94±0.2° 2-theta and 20.44±0.2° 2-theta.

Embodiment 48: The pharmaceutically acceptable form of embodiment 1, wherein the pharmaceutically acceptable form is a succinate form.

Embodiment 49: The pharmaceutically acceptable form of embodiment 48, wherein the succinate form comprises a succinate salt.

Embodiment 50: The pharmaceutically acceptable form of embodiment 48, wherein the succinate form comprises a succinate co-crystal.

Embodiment 51: A pharmaceutical composition comprising an amount of a pharmaceutically acceptable form of Compound 1 according to one any one of embodiments 1 to 50, and one or more pharmaceutically acceptable excipient.

Embodiment 52: A method of inhibiting CD73 activity in a cell comprising contacting CD73 in the cell with an effective amount of a pharmaceutically acceptable form of any one of embodiments 1 to 50.

Embodiment 53: A method of treating cancer in a subject, comprising administering to the subject a therapeutically effective amount of a pharmaceutically acceptable form of any one of embodiments 1 to 50.

Embodiment 54: A method of treating cancer in a subject, comprising administering to the subject a pharmaceutical composition of embodiment 51.

Embodiment 55: The method of embodiment 53 or 54, wherein the cancer is lung cancer, melanoma, breast cancer, ovarian cancer, colorectal cancer, gastric cancer, gallbladder cancer, prostate cancer, renal cancer, multiple myeloma, or a lymphoma.

Embodiment 56: The method of any one of embodiments 53 to 55, wherein the cancer expresses CD73.

Embodiment 57: The method of any one of embodiments 53 to 56, wherein CD73 is upregulated in the cancer to be treated.

Embodiment 58: The method of any one of embodiments 52 to 57, further comprising administering one or more second therapeutic agents.

Embodiment 59: The method of embodiment 58, wherein the second therapeutic agent is a chemotherapeutic agent or an immunotherapy agent.

Embodiment 60: A method of treating an infection in a subject, comprising administering to the subject a therapeutically effective amount of a pharmaceutically acceptable form of any one of embodiments 1 to 50.

Embodiment 61: A method of treating an infection in a subject, comprising administering to the subject a pharmaceutical composition of embodiment 51.

Embodiment 62: The method of embodiment 60 or 61, wherein the infection is a viral infection.

Embodiment 63: The method of embodiment 60 or 61, wherein the infection is a parasitic infection.

Embodiment 64: A method of treating a neurodegenerative disease in a subject, comprising administering to the subject a pharmaceutically acceptable form of any one of embodiments 1 to 50.

Embodiment 65: A method of treating a neurodegenerative disease in a subject, comprising administering to the subject a pharmaceutical composition of embodiment 51.

Embodiment 66: The method of embodiment 64 or 65, wherein the neurodegenerative disease is Alzheimer's disease, Parkinson's disease, Huntington's disease, schizophrenia, or autism.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutically acceptable form of Compound 1: 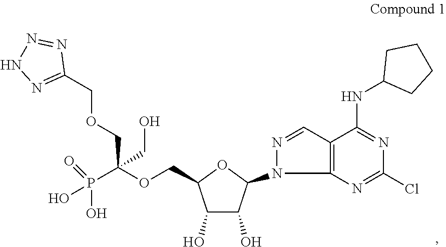 [SMILES: O=P(O)(O)[C@@](CO)(COCc1nn[nH]n1)OC[C@H]1O[C@@H](n2ncc3c(NC4CCCC4)nc(Cl)nc32)[C@H](O)[C@@H]1O]
wherein the pharmaceutically acceptable from is selected from a gentisate form and a succinate form.

2. The pharmaceutically acceptable form of claim 1, wherein the pharmaceutically acceptable form is a gentisate form.

3. The pharmaceutically acceptable form of claim 2, wherein in the gentisate form the molar ratio between Compound 1 and gentisic acid is about 1:1.

4. The pharmaceutically acceptable form of claim 3, wherein the gentisate form is a crystalline solid.

5. The pharmaceutically acceptable form of claim 4, wherein the crystalline solid exhibits a peak in an x-ray powder diffraction (XRPD) pattern at 9.25±0.2° 2-theta.

6. The pharmaceutically acceptable form of claim 5, wherein the crystalline solid exhibits further peaks in an x-ray powder diffraction (XRPD) pattern at 6.97±0.2° 2-theta, 20.53±0.2° 2-theta, and 26.08±0.2° 2-theta.

7. The pharmaceutically acceptable form of claim 6, wherein the crystalline solid exhibits further peaks in an x-ray powder diffraction (XRPD) pattern at 14.61±0.2° 2-theta and 18.89±0.2° 2-theta.

8. The pharmaceutically acceptable form of any one of claims 5 to 7, wherein the crystalline solid further comprises a peak in a differential scanning calorimetry pattern of from about 150 °C to about 170 °C.

9. The pharmaceutically acceptable form of any one of claims 1 to 8, wherein the pharmaceutically acceptable form exhibits less than about 10% degradation when the pharmaceutically acceptable form is stored at 25 °C and 60% relative humidity for at least 7 days.

10. The pharmaceutically acceptable form of claim 1, wherein the pharmaceutically acceptable form is a succinate form.

11. A pharmaceutical composition comprising an amount of a pharmaceutically acceptable form of Compound 1 according to one any one of claims 1 to 10, and one or more pharmaceutically acceptable excipient.

12. A method of inhibiting CD73 activity in a cell comprising contacting CD73 in the cell with an effective amount of a pharmaceutically acceptable form of any one of claims 1 to 10.

13. A method of treating cancer in a subject, comprising administering to the subject a therapeutically effective amount of a pharmaceutically acceptable form of any one of claims 1 to 10.

14. A method of treating cancer in a subject, comprising administering to the subject a pharmaceutical composition of claim 11.

15. The method of claim 13 or 14, wherein the cancer is lung cancer, melanoma, breast cancer, ovarian cancer, colorectal cancer, gastric cancer, gallbladder cancer, prostate cancer, renal cancer, multiple myeloma, or a lymphoma.
